(19) United States
(12) Patent Application Publication
(10) Pub. No.: US 2020/0362018 A1
(43) Pub. Date: Nov. 19, 2020

(54) INHIBITORS OF THE 20S PROTEASOME

(71) Applicant: Yeda Research and Development Co. Ltd., Rehovot (IL)

(72) Inventors: Michal SHARON, Rehovot (IL); Maya OLSHINA, Rehovot (IL); Fanindra Kumar DESHMUKH, Rehovot (IL); Dan S. TAWFIK, Rehovot (IL)

(73) Assignee: Yeda Research and Development Co. Ltd., Rehovot (IL)

(21) Appl. No.: 16/984,187

(22) Filed: Aug. 4, 2020

Related U.S. Application Data
(63) Continuation of application No. PCT/IL2019/050129, filed on Feb. 3, 2019.
(60) Provisional application No. 62/627,813, filed on Feb. 8, 2018.

(51) Int. Cl. C07K 14/81

(52) U.S. Cl. CPC C07K 14/81; A61K 38/00

(57) ABSTRACT

Polypeptide comprising a CATH 3.40 architecture, the architecture comprising an amino acid sequence as set forth in SEQ ID NO: 18, which are capable of specifically inhibiting the activity of a 20S proteasome are disclosed. Uses thereof are also disclosed.

### RELATED APPLICATIONS

This application is a US Continuation of PCT Patent Application No. PCT/IL2019/050129 having International filing date of Feb. 3, 2019, which claims the benefit of priority under 35 USC §119(e) of U.S. Provisional Patent Application No. 62/627,813 filed on Feb. 8, 2018. The contents of the above applications are all incorporated by reference as if fully set forth herein in their entirety.

### SEQUENCE LISTING STATEMENT

The ASCII file, entitled 83465SequenceListing.txt, created on Aug. 4, 2020, comprising 5,627 bytes, submitted concurrently with the filing of this application is incorporated herein by reference.

### FIELD AND BACKGROUND OF THE INVENTION

The present invention, in some embodiments thereof, relates to polypeptides that are capable of inhibiting the 20S proteasome.

Proteasomal protein degradation is crucial in maintaining cellular integrity and in regulating key cellular processes including cell cycle, proliferation and cell death. Proteasomal degradation is mediated mainly by two proteasomal complexes; the 26S proteasome, that consists of the 20S catalytic domain and two 19S regulatory particles (RP) and the 20S proteasome in isolation. In the well-characterized ubiquitin-proteasome system (UPS) a protein is targeted for degradation by specific modification by a set of enzymes that conjugates a poly-ubiquitin chain to the protein. The poly-ubiquitinated substrate is then recognized by specific subunits of the 19S RP of the 26S proteasome where it is de-ubiquitinated, unfolded by the ATPases and translocated into the 20S catalytic chamber for degradation. Recently an ubiquitin-independent proteasomal degradation pathway has been described whereby intrinsically disordered proteins (IDPs) such as p53, c-FOS, BimEL and others can be degraded by the 20S proteasome in a process that does not involve active ubiquitin tagging. The 20S proteasome has been also shown to be activated by the REG (11S) family members inducing the degradation of SRC-3, p21 and other proteins. Thus, there are at least two distinct proteasomal protein degradation pathways, each regulated by the distinct 26S and 20S proteasomal complexes.

To date, proteasome inhibitors, such as bortezomib and carfilzomib have been developed for treating certain cancers, especially multiple myeloma and mantle cell lymphoma, and many other such inhibitors are currently being tested for anti-tumor and anti-inflammatory activities as well as for treating auto-immune diseases. These drugs, however, target the chymotrypsin-like activity of the 20S proteasome, and inhibit the activities of both the 20S and 26S proteasomes. Thus, it possible that selective drug intervention specifically inhibiting the 20S proteasomes will improve the rates of cancer cell toxicity, and/or minimize the deleterious side effects of the current therapeutic regimens and expand their therapeutic applications.

Background art includes Moscovitz et al., Nature Communications 6, 6609, doi: 10.1038/ncomms7609(2015).

### SUMMARY OF THE INVENTION

According to an aspect of some embodiments of the present invention there is provided an isolated polypeptide comprising a CATH 3.40 architecture, the architecture comprising an amino acid sequence as set forth in SEQ ID NO: 18, wherein the polypeptide comprises a modification such that is shows enhanced bioavailability and/or efficacy in vivo as compared to the same polypeptide lacking the modification, the polypeptide capable of specifically inhibiting the activity of a 20S proteasome.

According to an aspect of some embodiments of the present invention there is provided an isolated polypeptide being a C-terminal truncation mutant of a protein selected from the group consisting of DJ-1, NQO1, NQO2, CBR3, PGDH, RBBP9, NRas, KRas, HRas, RhoA, RhoB, RhoC, Rap1A, Rap1B, Rap2A, ETFB and PGAM1, the polypeptide capable of specifically inhibiting the activity of a 20S proteasome.

According to an aspect of some embodiments of the present invention there is provided an isolated polypeptide comprising a CATH 3.40 architecture, the architecture comprising an amino acid sequence as set forth in SEQ ID NO: 18, wherein the polypeptide is no longer than 250 amino acids, the polypeptide capable of specifically inhibiting the activity of a 20S proteasome.

According to an aspect of some embodiments of the present invention there is provided an isolated polypeptide comprising a CATH 3.40 architecture, the architecture comprising an amino acid sequence as set forth in SEQ ID NO: 18, wherein the polypeptide is attached to a cell penetrating moiety, the polypeptide capable of specifically inhibiting the activity of a 20S proteasome.

According to an aspect of some embodiments of the present invention there is provided an isolated polynucleotide encoding the polypeptide described herein.

According to an aspect of some embodiments of the present invention there is provided a pharmaceutical agent comprising the isolated polypeptide described herein or the isolated polynucleotide of claim 19 as the active agent and a pharmaceutically acceptable carrier.

According to an aspect of some embodiments of the present invention there is provided the isolated polypeptide described herein, for use in treating a disease a disease for which inhibiting a 20S proteasome is advantageous.

According to an aspect of some embodiments of the present invention there is provided an isolated polypeptide comprising a CATH 3.40 architecture which comprises the sequence as set forth in SEQ ID NO: 18 for use in treating a disease a disease for which inhibiting a 20S proteasome is advantageous, with the proviso that the isolated polypeptide is not full length DJ-1 or NQO1.

According to an aspect of some embodiments of the present invention there is provided a method of treating a disease for which inhibiting a 20S proteasome is advantageous in a subject in need thereof, the method comprising administering to the subject a therapeutically effective amount of the isolated polypeptide described herein, thereby treating the disease.

According to an aspect of some embodiments of the present invention there is provided a method of treating a disease for which inhibiting a 20S proteasome is advantageous in a subject in need thereof, the method comprising administering to the subject a therapeutically effective amount of an isolated polypeptide comprising a CATH 3.40 architecture, the architecture comprising the amino acid sequence as set forth in SEQ ID NO: 18, with the proviso that the isolated polypeptide is not full length DJ-1 or NQO1.

According to embodiments of the present invention the isolated polypeptide is a C-terminal truncation mutant of a protein selected from the group consisting of DJ-1, NQO1, NQO2, CBR3, PGDH, RBBP9, NRas, KRas, HRas, RhoA, RhoB, RhoC, Rap1A, Rap1B, Rap2A, ETFB and PGAM1.

According to embodiments of the present invention the polypeptide is truncated at the C-terminus by at least 100 amino acids.

According to embodiments of the present invention, the isolated polypeptide is no longer than 300 amino acids.

According to embodiments of the present invention, the isolated polypeptide comprises a modification such that is shows enhanced bioavailability and/or efficacy in vivo as compared to the same polypeptide lacking the modification.

According to embodiments of the present invention, the modification comprises a chemical modification.

According to embodiments of the present invention, the isolated polypeptide is attached to a heterologous polypeptide.

According to embodiments of the present invention, the heterologous polypeptide is selected from the group consisting of human serum albumin, immunoglobulin and transferrin.

According to embodiments of the present invention the immunoglobulin comprises an Fc domain.

According to embodiments of the present invention, the isolated polypeptide is attached to a cell penetrating moiety.

According to embodiments of the present invention, the cell penetrating moiety comprises a cell penetrating peptide.

According to embodiments of the present invention, the architecture comprises a sequence selected from the group consisting of 1-17.

According to embodiments of the present invention the isolated polypeptide is a C-terminal truncation mutant of a protein selected from the group consisting of NQO2, CBR3, PGDH, RBBP9, NRas, KRas, HRas, RhoA, RhoB, RhoC, Rap1A, Rap1B, Rap2A, ETFB and PGAM1.

According to embodiments of the present invention, the isolated polypeptide is a recombinant polypeptide.

According to embodiments of the present invention, the isolated polypeptide is capable of binding to the 20S proteasome.

According to embodiments of the present invention, the disease is cancer.

According to embodiments of the present invention, the disease is an autoimmune disease.

According to embodiments of the present invention, the disease is a neurodegenerative disease.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B illustrate the functional conservation of DJ-1 across evolution. (A) Degradation of α-synuclein (α-Syn) by the R. norvegicus (Mammalian) 20S proteasome in the presence of DJ-1 homologues from human, S. cerevisiae (Yeast) and T. acidophilum (Archaea). At the indicated time points, aliquots were quenched and evaluated by SDS-PAGE. All species of DJ-1 homologues inhibited the function of the mammalian 20S proteasome. (B) Degradation of α-Syn by the archaeal, yeast and mammalian 20S proteasomes in the presence of human DJ-1. Human DJ-1 inhibited 20S proteasomes from all tested species.

FIGS. 2A-C illustrate that human DJ-1 physically binds to the 20S proteasome from T. acidophilum. Free 20S proteasomes (A), 20S proteasomes mixed with DJ-1 (B) and free DJ-1 (C) were examined by native MS. For each sample, the most intense charge state obtained in the MS spectrum was subjected to MS/MS analysis (inset shows the MS spectrum of the free 20S proteasome; the 73⁺ charge state highlighted in red was subjected to MS/MS analysis). Comparison of the free 20S spectrum (A), with that recorded for 20S in the presence of DJ-1 (B), revealed additional peaks that correspond in mass to the monomeric form of DJ-1, as seen in (C). By extrapolation, we can therefore conclude that prior to MS/MS analysis, human DJ-1 binds to the 20S proteasome from T. acidophilum. Blue dots correspond to the α-subunit of the 20S proteasome; yellow dots represent monomers of DJ-1.

FIGS. 3A-B illustrates that NQO2, CBR3, PGDH, NRas, KRas and RhoA inhibit the 20S proteasome. To examine whether the putative 20S regulators can protect substrates from 20S proteasome proteolysis, a series of time-dependent degradation assays was performed using the intrinsically unstructured protein α-synuclein. As a control, the proteasome inhibitor MG132 was used. At the indicated time points, aliquots were quenched and evaluated by SDS-PAGE followed by quantitative image analysis (FIGS. 3A, B). α-synuclein was stable in the absence of the 20S proteasome; however, after its addition, it was degraded. In the presence of NQO2, CBR3, PGDH, NRas, KRas and RhoA, however, there was a marked decrease in the degradation rate.

FIGS. 4A-C illustrate that 20S PIPs physically bind to the 20S proteasome. Samples were sprayed under native conditions followed by isolation of peaks corresponding to the 20S proteasome (panel A, inset) and subsequent MS/MS analysis. Comparison of the MS/MS spectra of (A) 20S proteasome alone, (B) 20S proteasome incubated with CBR3, or (C) incubated with NRas reveal the dissociation of intact alpha-subunits of the 20S proteasome (blue balls) and the regulators (CBR3—green balls, NRas—pink balls), demonstrating that they physically bind to the 20S proteasome. The measured molecular weights indicated agree with the predicted size of monomeric CBR3 (30937.3 Da) and monomeric NRas (19603.3 Da).

FIGS. 5A-B illustrate the functional conservation of CBR3 across evolution. (A) Degradation of α-synuclein (α-Syn) by the S. cerevisiae (Yeast) and (B) T. acidophilum (Archaea) 20S proteasomes. Human CBR3 inhibited 20S proteasomes from all tested species. At the indicated time points, aliquots were quenched and evaluated by SDS-PAGE.

FIGS. 6A-B illustrates that PGDH and CBR3 bind the 20S proteasome. HEK293 cells stably expressing FLAG tagged β4 subunit of the 20S proteasome were lysed and subjected to immunoprecipitation with anti-PGDH and anti-FLAG antibodies, followed by Western blot analysis. The total protein load (L), unbound fraction (UB) and immunoprecipitated fraction (IP) were run in parallel. The presence of PGDH and CBR3 in the IP fraction of the FLAG IP (FIG. 6A lower panel, FIG. 6B middle right panel)), and FLAG-20S proteasome in the IP fraction of the aPGDH and aCBR3 IPs (FIGS. 6A and B upper panels) indicates binding of PGDH and CBR3 to the 20S proteasome. Immunoblot analysis using an antibody directed towards Rpn2 (FIG. 6B), did not give rise to a band when aCBR3 was used for pull down, indicating that CBR3 binds to the 20S, but not 26S, proteasome.

FIGS. 7A-B illustrate that NQO2 and NRas stabilize the cellular levels of 20S proteasome substrates. HEK293 cells were transiently transfected to silence NQO2 (A) and NRas (B). As a control, non-targeting siRNA (NT) was used. Cells were lysed and cell extracts were loaded onto SUS-PAGE gel and analyzed by western blot using the indicated antibodies. The results indicate that silencing NQO2 and NR as reduces the cellular levels of full length p53, a 20S proteasome substrate. Δ40p53 levels, which results from 20S mediated cleavage of p53, were increased. The addition of the proteasome inhibitor, MG132. reduced Δ40p53 formation.

FIGS. 8A-D illustrate that CBR3, NQO2, PGDH and NRas stabilize the cellular levels of 20S proteasome substrates. HEK293 cells were transiently transfected to overexpress CBR3 (A), NQO2 (B) and PGDH(C). 108T melanoma cells were transiently transfected to overexpress NRas (D). As a control, GFP was overexpressed in parallel with each experiment. Cells were lysed and cell extracts were loaded on SDS-PAGE and analyzed by western blot with the indicated antibodies. The results indicate that overexpressing CBR3, NQO2 and PGDH all stabilize the levels of full-length p53 (FIGS. 8A-C), while α-synuclein levels are stabilized by CBR3 (FIG. 8A) and NRas (FIG. 8D) overexpression, indicating inhibition of the 20S proteasome.

FIG. 9 is a cryo-electron microscopy (Cryo-EM) reconstruction, which suggests that the catalytic core regulator (CCR) CBR3 binds to the β-subunit of the 20S proteasome. The structure of the human 20S proteasome (4R3O, cyan) was fit into the electron density map. The extra electron density near the β₄-subunit (magenta), reveals the binding site of CBR3 (green).

FIG. 10 illustrates that CCRs bind the 20S proteasome β-ring. Peptide array screening revealed that CCR's—CBR3 and NQO1 both bind to a β strand-loop-βstrand secondary structure (in red) within the β-subunit ring of the T. acidophilum archaeal 20S proteasome (grey, 1PMA). The zoom-in image represents a single β-subunit.

FIGS. 11A-D illustrate that an internal β-strand within the β-sheet core of the CCRs Rossmann fold binds the 20S proteasome. The peptide array results revealed that 20S proteasomes from archaea, yeast and human cells consensually bind a β-strand within the core β-sheet of the Rossmann fold (in red) (A) human DJ-1 (1UCF), (B) archaeal DJ-1, (C) NQO1 (1D4A) and (D) CBR3 (2HRB). The web interface of protein Homolgy/Analogy Recognition Engine Phyre2 portal was used for generating the structure of archaeal DJ-1.

FIGS. 12A-B illustrate that CCRs are not degraded by the 20S proteasome. (A) In vitro degradation assays of each CCR with 20S proteasome in the absence of α-synuclein. As controls, α-synuclein alone and in the presence of 20S proteasome (top two panels) is included to ensure active 20S proteasome. (B) Quantification of α-synuclein (from control panels in A) or each CCR from three independent experiments. Error bars represent S.E.M.

FIG. 13 illustrates that native MS does not detect any interactions between the α-synuclein substrate and CCRs. α-synuclein was analysed by native mass spectrometry either alone (top panel) or in the presence of each of the CCRs. The charge series corresponding to α-synuclein were measured in each spectrum (gray balls). Each of the CCRs were detected in their respective spectra (NQO2—yellow, CBR3—lime, PGDH—green, NRas—dark purple, KRas—dark blue, RhoA—teal). No larger molecular weight complexes were detected in any of the spectra, indicating that α-synuclein does not bind to any of the CCRs.

FIGS. 14A-J illustrate that CCRs physically bind the 20S proteasome. 20S proteasomes alone or in the presence of CCRs were analysed by native MS to determine the binding status of the CCRs to the 20S proteasome. (A) Native MS spectrum of 20S proteasomes. Highlighted peaks were isolated and subjected to increased collision energy. (B) MS/MS spectrum of 20S proteasome, peak series of individual dissociated 20S subunits were identified (white, grey, black balls). (C-J) 20S proteasomes were pre-incubated with CCRs, followed by MS/MS analysis to identify CCR binding. Unique peak series corresponding in size to the monomeric size of each of the CCRs was detected (colored balls), indicating CCRs physically bind to the 20S proteasome.

FIGS. 15A-H illustrate that CCRs bind to the 20S proteasome in cells. HA-tagged (A) NQO2, (B) PGDH, (C) NRas and (D) RhoA were overexpressed in HEK293 cells stably expressing FLAG-tagged B4 subunit of the 20S proteasome. In (D), cells were exposed to 100 uM DEM for 48 hrs prior to collection and lysis. Lysates were subjected to IP using either anti-FLAG-affinity gel, anti-HA or anti-Rpn2 antibodies, or Protein G beads as a control. Total staring lysate (L), unbound proteins (UB) and IP samples were analysed by Western blot using anti-205, anti-HA or anti-Rpn2 antibodies. Bands corresponding to HA (i.e. CCRs) in the FLAG (20S) IP and FLAG (20S) in the HA IP were quantified and compared with their Protein G equivalents (E, F, G, H). Each of the CCRs were significantly enriched in the FLAG (20S) IP demonstrating that the CCRs bind to the 20S proteasome. The reciprocal IP with anti-HA confirmed this interaction, with significant enrichment of the FLAG (20S) bands. Quantifications demonstrate the average of (E, F, H) four or (G) five independent experiments. Band intensity measurements were subjected to Students t-test analysis, * p<0.05, ** p<0.01, *** p<0.001, **** p<0.0001. Error bars represent S.E.M.

FIG. 16 illustrates that CCRs inhibit the degradation of partially folded proteins by the 20S proteasome. In vitro degradation assays of each CCR with substrates (Sub) α-synuclein (left), or OxCalmodulin (right). MG132 was included as a control for 20S proteasome inhibition. Panels labelled with an asterisk are immunoblots using anti-calmodulin antibody of the degradation assays with OxCalmodulin for those CCRs that are the same size as the substrate: RBBP9, NRas, KRas, HRas and RhoA. Quantification of three independent experiments is displayed below the gel images, error bars represent S.E.M.

### DESCRIPTION OF SPECIFIC EMBODIMENTS OF THE INVENTION

The present invention, in some embodiments thereof, relates to polypeptides that are capable of inhibiting the 20S proteasome.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

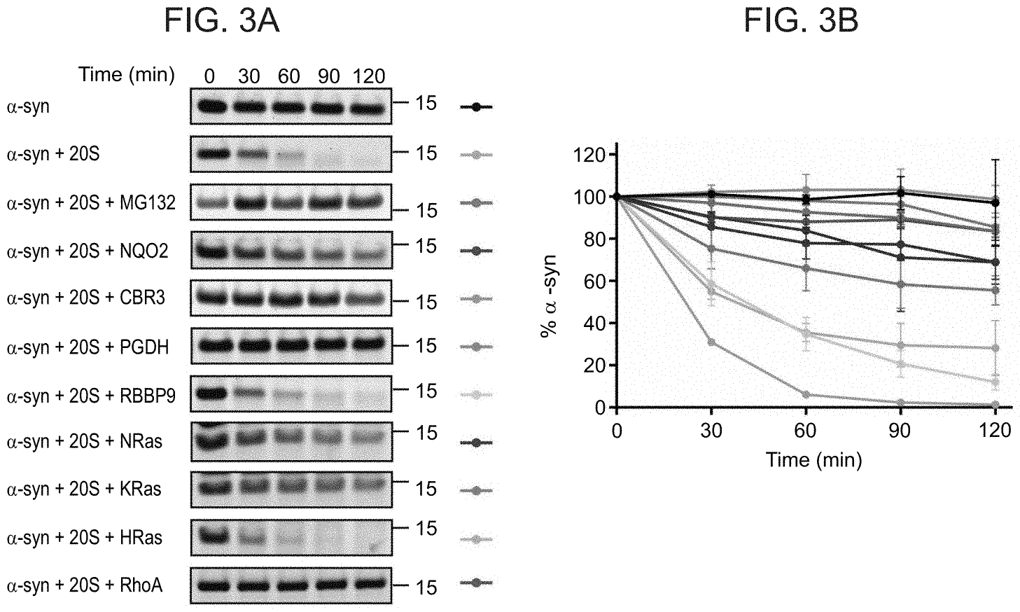

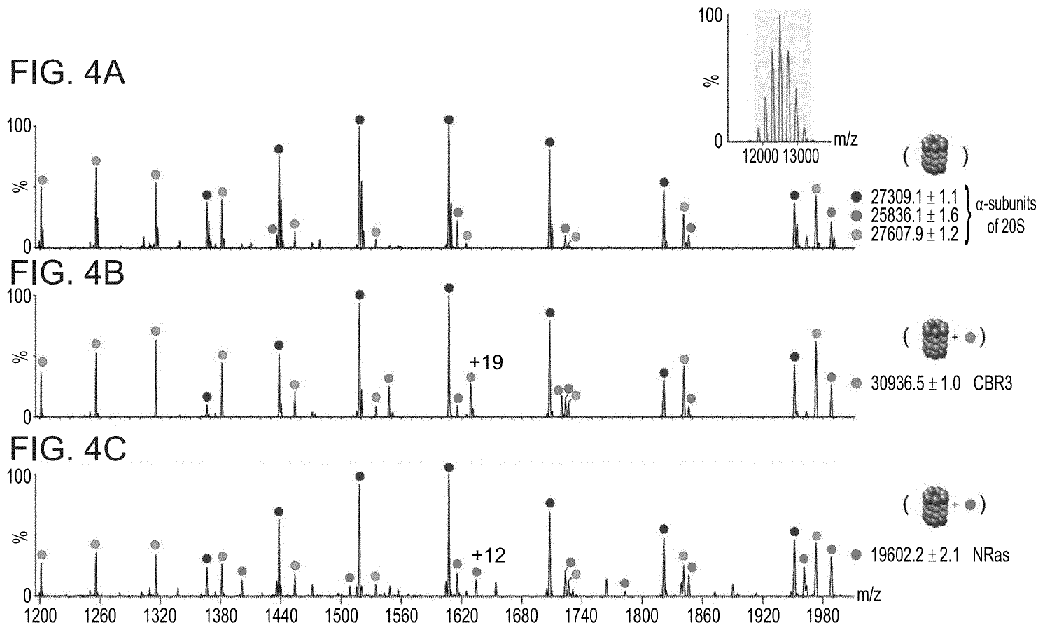

The present inventors have identified an N-terminal sequence motif, which is comprised in a structural fold that is critical for 20S proteolysis inhibition. The present inventors have identified a family of human proteins harboring this motif. Whilst reducing the present invention to practice, the present inventors showed that these proteins indeed inhibit the 20S proteasome function (see FIGS. 3A-B). Furthermore, the present inventors showed that these proteins were able to specifically bind to the 20S proteasome (rather than the 26S proteasome; see FIGS. 4A-C and 6A-B).

Taken together, the present results indicate that polypeptides harboring the uncovered motif can be used to treat diseases for which inhibiting the proteasome 20S is advantageous.

Thus, according to a first aspect of the present invention, there is provided an isolated polypeptide comprising a CATH architecture ID 3.40, the CATH architecture comprising an amino acid sequence as set forth in SEQ ID NO: 18 [(K/R)₁₋₂(V/L/I/A)₄], wherein the polypeptide is capable of specifically inhibiting the activity of a 20S proteasome.

According to another aspect of the present invention there is provided isolated polypeptide being a C-terminal truncation mutant of a protein which comprises a CATH architecture ID 3.40, said CATH architecture comprising an amino acid sequence as set forth in SEQ ID NO: 18 i.e. (K/R)₁₋₂(V/L/I/A)₄, the polypeptide capable of specifically inhibiting the activity of a 20S proteasome.

The term “polypeptide” as used herein refers to a polymer of natural or synthetic amino acids, encompassing native peptides (either degradation products, synthetically synthesized polypeptides or recombinant polypeptides) and peptidomimetics (typically, synthetically synthesized peptides), as well as peptoids and semipeptoids which are polypeptide analogs.

The phrase “CATH architecture ID 3.40” refers to a structure composed of alternating beta strands and alpha helical segments where the beta strands are hydrogen bonded to each other forming an extended beta sheet and the alpha helices surround both faces of the sheet to produce a three-layered sandwich—i.e. α/β/α sandwich with parallel β-sheet core. In one embodiment, the CATH architecture ID 3.40 refers to a Rossmann fold. In another embodiment, the CATH architecture ID 3.40 refers to a P-loop_NTPase structure.

Methods of identifying whether a polypeptide comprises such a structure are known in the art and include X-ray crystallography and NMR.

The polypeptide of this aspect of the present invention further comprises a sequence motif as set forth in SEQ ID NO: 18. In a particular embodiment, the sequence motif is set forth in SEQ ID NO: 19. The sequence motif is at a position such that it is comprised in the CATH 3.40 architecture.

According to a particular embodiment, the sequence motif as set forth in SEQ ID NO: 18 is present at a position, which is no more than 15 amino acids away from the N terminus of the polypeptide. According to a particular embodiment, the sequence motif as set forth in SEQ ID NO: 18 is present at a position, which is no more than 14 amino acids away from the N terminus of the polypeptide. According to a particular embodiment, the sequence motif as set forth in SEQ ID NO: 18 is present at a position, which is no more than 13 amino acids away from the N terminus of the polypeptide. According to a particular embodiment, the sequence motif as set forth in SEQ ID NO: 18 is present at a position, which is no more than 12 amino acids away from the N terminus of the polypeptide. According to a particular embodiment, the sequence motif as set forth in SEQ ID NO: 18 is present at a position, which is no more than 11 amino acids away from the N terminus of the polypeptide. According to a particular embodiment, the sequence motif as set forth in SEQ ID NO: 18 is present at a position, which is no more than 10 amino acids away from the N terminus of the polypeptide. According to a particular embodiment, the sequence motif as set forth in SEQ ID NO: 18 is present at a position, which is no more than 9 amino acids away from the N terminus of the polypeptide. According to a particular embodiment, the sequence motif as set forth in SEQ ID NO: 18 is present at a position, which is no more than 8 amino acids away from the N terminus of the polypeptide. According to a particular embodiment, the sequence motif as set forth in SEQ ID NO: 18 is present at a position which is no more than 7 amino acids away from the N terminus of the polypeptide. According to a particular embodiment, the sequence motif as set forth in SEQ ID NO: 18 is present at a position, which is no more than 6 amino acids away from the N terminus of the polypeptide. According to a particular embodiment, the sequence motif as set forth in SEQ ID NO: 18 is present at a position, which is no more than 5 amino acids away from the N terminus of the polypeptide. According to a particular embodiment, the sequence motif as set forth in SEQ ID NO: 18 is present at a position, which is no more than 4 amino acids away from the N terminus of the polypeptide. According to a particular embodiment, the sequence motif as set forth in SEQ ID NO: 18 is present at a position, which is no more than 3 amino acids away from the N terminus of the polypeptide. According to a particular embodiment, the sequence motif as set forth in SEQ ID NO: 18 is present at a position, which is no more than 2 amino acids away from the N terminus of the polypeptide. It will be appreciated that when the polypeptide comprises the sequence motif as set forth in SEQ ID NO: 19, the motif is present at the N terminus of the polypeptide.

Examples of amino acid sequences, which harbor the sequence motif as set forth in SEQ ID NO: 19, which can be comprised in the polypeptides of the present invention are set forth in SEQ ID NOs: 1-17.

Exemplary proteins that comprise the sequence motif as set forth in SEQ ID NO: 19, in a CATH 3.40 architecture include, but are not limited to DJ-1, NQO1, NQO2, CBR3, PGDH, RBBP9, NRas, KRas, HRas, RhoA, RhoB, RhoC, Rap1A, Rap1B, Rap2A, ETFB and PGAM1.

It will be appreciated that the polypeptides of this aspect of the present invention are not full-length wild-type sequences of the above described proteins.

Thus, the polypeptides of this aspect of the present invention may be a C terminal truncation mutant (i.e. truncated at the C terminus) of one of the above described proteins (or another protein known to comprise the sequence motif of SEQ ID NO: 18 in a CATH 3.40 architecture).

In one embodiment, the polypeptide is truncated at the C terminus of the corresponding wild-type polypeptide by at least 5 amino acids, 10 amino acids, 15 amino acids, 20 amino acids, 25 amino acids, 30 amino acids, 35 amino acids, 40 amino acids, 45 amino acids, 50 amino acids, 55 amino acids, 60 amino acids, 65 amino acids, 70 amino acids, 75 amino acids, 80 amino acids, 85 amino acids, 90 amino acids, 95 amino acids, 100 amino acids, 105 amino acids, 110 amino acids, 115 amino acids, 120 amino acids, 125 amino acids, 130 amino acids, 135 amino acids, 140 amino acids, 145 amino acids or 150 amino acids or more.

In another embodiment, the polypeptide of this aspect of the present invention is truncated (preferably at the C terminus) such that its length is no more than 90%, 85%, 80%, 75%, 70%, 65%, 60%, 55% or 50% of the length of the corresponding wild-type amino acid sequence.

Altogether, the polypeptides of this aspect of the present invention may comprise between 50-500, 50-450, 50-400, 50-350, 50-300, 50-250, 50-200, 50-150, 50-100 amino acids of a protein known to comprise the above described sequence motif in a CATH 3.40 architecture.

According to still another embodiment, the polypeptides of this aspect of the present invention may comprise between 50-500, 50-450, 50-400, 50-350, 50-300, 50-250, 50-200, 50-150 or 50-100 amino acids.

It will be appreciated that the polypeptide of this aspect of the present invention may comprise a modification such that is shows enhanced bioavailability and/or efficacy in vivo as compared to the same polypeptide lacking the modification.

Additionally, or alternatively, the polypeptides of this aspect of the present invention may have modifications rendering them even more stable in vivo or more capable of penetrating into cells.

Such modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc.).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables 1 and 2 below list naturally occurring amino acids (Table 1) and non-conventional or modified amino acids (Table 2) which can be used with the present invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl)glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α- | Aabu | N-methyl-α-amino-α- | Nmaabu |
| methylbutyrate | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | a-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2- | Nnbhm |
| diphenylethyl)glycine | | diphenylethyl)carbamylmethyl- | |
| | | glycine | |
| N-(3,3- | Nbhe | N-(N-(3,3- | Nnbhe |
| diphenylpropyl)glycine | | diphenylpropyl)carbamylmethyl- | |
| | | glycine | |
| 1-carboxy-1-(2,2- | Nmbe | 1,2,3,4- | Tic |
| diphenyl | | tetrahydroisoquinoline-3- | |
| ethylamino)cyclopropane | | carboxylic acid | |
| phospho serine | pSer | phospho threonine | PThr |
| phospho tyrosine | pTyr | O-methyl-tyrosine | |
| 2-aminoadipic acid | | hydroxylysine | |

The amino acids of the polypeptides of the present invention may be substituted either conservatively or non-conservatively compared to the wild-type sequences of proteins which comprise a CATH 3.40 architecture, said fold comprising an amino acid sequence as set forth in SEQ ID NO: 18 i.e. (K/R)₁₋₂(V/L/I/A)₄.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[—CH₂)₅₋COOH]CO— for aspartic acid. Those non-conservative substitutions which fall under the scope of the present invention are those which still constitute a peptide having anti-bacterial properties.

According to a specific embodiment, the polypeptides of this aspect of the present invention are no more than 50%, 55%, 60%, 70%, 75%, 80%, 85%, 90%, 91%, 92%, 93%, 94%, 95%, 96% homologous or identical to the sequences of their corresponding full length, wild-type sequences.

As mentioned, the N and C termini of the polypeptides of the present invention may be protected by functional groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

These moieties can be cleaved in vivo, either by hydrolysis or enzymatically, inside the cell. Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—phenyl-O—CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH2, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

The polypeptides of the present invention may also comprise non-amino acid moieties, such as for example, hydrophobic moieties (various linear, branched, cyclic, polycyclic or heterocyclic hydrocarbons and hydrocarbon derivatives) attached to the peptides; non-peptide penetrating agents; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

Attaching the amino acid sequence component of the peptides of the invention to other non-amino acid agents may be by covalent linking, by non-covalent complexion, for example, by complexion to a hydrophobic polymer, which can be degraded or cleaved producing a compound capable of sustained release; by entrapping the amino acid part of the peptide in liposomes or micelles to produce the final peptide of the invention. The association may be by the entrapment of the amino acid sequence within the other component (liposome, micelle) or the impregnation of the amino acid sequence within a polymer to produce the final peptide of the invention.

It will be appreciated that the polypeptide of some embodiments of the invention may be chemically modified following expression for increasing bioavailability.

Thus, for example, the present invention contemplates modifications wherein polypeptide is linked to a polymer. The polymer selected is usually modified to have a single reactive group, such as an active ester for acylation or an aldehyde for alkylation, so that the degree of modification may be controlled. Included within the scope of polymers is a mixture of polymers. Preferably, for therapeutic use of the end-product preparation, the polymer will be pharmaceutically acceptable.

The polymer or mixture thereof may be selected from the group consisting of, for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol. In further still embodiments, the polypeptide is modified by PEGylation, HESylation CTP (C terminal peptide), crosslinking to albumin, encapsulation, modification with polysaccharide and polysaccharide alteration. The modification can be to any amino acid residue in the polypeptide.

According to one embodiment the modification is to the N or C-terminal amino acid of the polypeptide. This may be effected either directly or by way coupling to the thiol group of a cysteine residue added to the N or C-terminus or a linker added to the N or C-terminus such as Ttds. In further embodiments, the N or C-terminus of the polypeptide comprises a cysteine residue to which a protecting group is coupled to the N-terminal amino group of the cysteine residue and the cysteine thiolate group is derivatized with a functional group such as N-ethylmaleimide, PEG group, HESylated CTP.

It is well known that the properties of certain proteins can be modulated by attachment of polyethylene glycol (PEG) polymers, which increases the hydrodynamic volume of the protein and thereby slows its clearance by kidney filtration. (See, for example, Clark et al., J. Biol. Chem. 271: 21969-21977 (1996). Therefore, it is envisioned that the core peptide residues can be PEGylated to provide enhanced therapeutic benefits such as, for example, increased efficacy by extending half-life in vivo. Thus, PEGylating the polypeptide will improve the pharmacokinetics and pharmacodynamics of the polypeptide.

PEGylation methods are well known in the literature and described in the following references, each of which is incorporated herein by reference: Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., Int. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C.sub.1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG.sub.2-NHS-40k (Nektar); mPEG₂-MAL-40k (Nektar), SUNBRIGHT GL2-400MA ((PEG).sub.240 kDa) (NOF Corporation, Tokyo), SUNBRIGHT ME-200MA (PEG20kDa) (NOF Corporation, Tokyo). The PEG groups are generally attached to the polypeptide via acylation, amidation, thioetherification or reductive alkylation through a reactive group on the PEG moiety (for example, an aldehyde, amino, carboxyl or thiol group) to a reactive group on the polypeptide (for example, an aldehyde, amino, carboxyl or thiol group).

The PEG molecule(s) may be covalently attached to any Lys or Cys residue at any position in the polypeptide. Other amino acids that can be used are Tyr and His. Optional are also amino acids with a Carboxylic side chain. The polypeptide described herein can be PEGylated directly to any amino acid at the N-terminus by way of the N-terminal amino group. A “linker arm” may be added to the polypeptide to facilitate PEGylation. PEGylation at the thiol side-chain of cysteine has been widely reported (See, e.g., Caliceti & Veronese, Adv. Drug Deliv. Rev. 55: 1261-77 (2003)). If there is no cysteine residue in the polypeptide, a cysteine residue can be introduced through substitution or by adding a cysteine to the N-terminal amino acid. Other options include reagents that add thiols to polypeptides, such as Traut's reagents and SATA.

In particular aspects, the PEG molecule is branched while in other aspects, the PEG molecule may be linear. In particular aspects, the PEG molecule is between 1 kDa and 150 kDa in molecular weight. More particularly, the PEG molecule is between 1 kDa and 100 kDa in molecular weight. In further aspects, the PEG molecule is selected from 5, 10, 20, 30, 40, 50 and 60 kDa.

A useful strategy for the PEGylation of the polypeptide consists of combining, through forming a conjugate linkage in solution, a peptide, and a PEG moiety, each bearing a special functionality that is mutually reactive toward the other. The polypeptide can be easily prepared by recombinant means as described above.

According to one embodiment, the PEG is “preactivated” prior to attachment to the polypeptide. For example, carboxyl terminated PEGs may be transformed to NHS esters for activation making them more reactive towards lysines and N-terminals.

According to another embodiment, the polypeptide is “preactivated” with an appropriate functional group at a specific site. Conjugation of the polypeptide with PEG may take place in aqueous phase or organic co-solvents and can be easily monitored by SDS-PAGE, isoelectric focusing (IEF), SEC and mass spectrometry. The PEGylated polypeptide is then purified. Small PEGs may be removed by ultra-filtration. Larger PEGs are typically purified using anion chromatography, cation chromatography or affinity chromatography. Characterization of the PEGylated polypeptide may be carried out by analytical HPLC, amino acid analysis, IEF, analysis of enzymatic activity, electrophoresis, analysis of PEG:protein ratio, laser desorption mass spectrometry and electrospray mass spectrometry.

Removal of excess free PEG may be performed by packing a column (Tricorn Empty High-Performance Columns, GE Healthcare) with POROS 50 HQ support (Applied Biosystems), following which the column is equilibrated with equilibration buffer (25 mM Tris-HCl buffer, pH 8.2). The PEGylated polypeptide is loaded onto the equilibrated column and thereafter the column is washed with 5CV of equilibration buffer. Under these conditions, the polypeptide binds to the column. PEGylated polypeptide is eluted in the next step by the elution buffer (0.3M NaCl, 25 mM Tris-HCl buffer, pH 8.2). The peak of this stage may be pooled and stored at 2-8° C. for short term, or frozen at −20 ° C. for long term storage.

Additionally, or alternatively, the polypeptides described herein may be attached to a cell penetrating agent.

As used herein the phrase “penetrating agent” refers to an agent which enhances translocation of an attached polypeptide across a cell membrane.

According to one embodiment, the penetrating agent is a peptide and is attached to the C or N terminus of the polypeptide (either directly or non-directly) via a peptide bond.

Typically, cell penetrating peptides have an amino acid composition containing either a high relative abundance of positively charged amino acids such as lysine or arginine, or have sequences that contain an alternating pattern of polar/charged amino acids and non-polar, hydrophobic amino acids.

Examples of cell penetrating peptides (CPPs) include long and short versions of TAT (YGRKKRR—SEQ ID NO: 20 and YGRKKRRQRRR—SEQ ID NO: 21) and PTD (RRQRR—SEQ ID NO: 22). By way of non-limiting example, cell penetrating peptide (CPP) sequences may be used in order to enhance intracellular penetration. Other contemplated CPPs may include:

GRKKRRQRRRPPQ—SEQ ID NO: 23;

GRKKRRQRRRPP—SEQ ID NO: 24;

GRKKRRQRRRP—SEQ ID NO: 25;

GRKKRRQRRR—SEQ ID NO: 26;

GRKKRRQRR—SEQ ID NO: 27;

GRKKRRQR—SEQ ID NO: 28;

GRKKRRQ—SEQ ID NO: 29;

According to a particular embodiment, the polypeptides of the present invention are attached to the cell penetrating peptides via a linking moiety.

Examples of linking moieties include but are not limited to a simple covalent bond, a flexible peptide linker, a disulfide bridge or a polymer such as polyethylene glycol (PEG). Peptide linkers may be entirely artificial (e.g., comprising 2 to 20 amino acid residues independently selected from the group consisting of glycine, serine, asparagine, threonine and alanine) or adopted from naturally occurring proteins. Disulfide bridge formation can be achieved, e.g., by addition of cysteine residues, as further described herein below.

Selection of the link between the two peptides should take into account that the link should not substantially interfere with the ability of the polypeptides of the present invention to inhibit the 20S proteasome (or to bind to the 20S proteasome) or the ability of the cell penetrating peptide to traverse the cell membrane.

Thus, for example, the linking moiety is optionally a moiety which is covalently attached to a side chain, an N-terminus or a C-terminus of the polypeptide of the present invention, as well as to a side chain, an N-terminus or a C-terminus of the cell penetrating peptide.

The linking moiety may be attached to the C-terminus of the polypeptide and to the N-terminus of the cell penetrating peptide.

Alternatively, the linking moiety may be attached to the N-terminus of the polypeptide peptide and to the C-terminus of the cell penetrating peptide.

The linker is preferably made up of amino acids linked together by peptide bonds. Thus, in preferred embodiments, the linker is made up of from 1 to 10 amino acids linked by peptide bonds, wherein the amino acids are selected from the 20 naturally occurring amino acids. Some of these amino acids may be glycosylated, as is well understood by those in the art.

In a more preferred embodiment, besides serine and glutamic acid the amino acids in the linker are selected from glycine, alanine, proline, asparagine and lysine. Even more preferably, besides serine and glutamic acid, the linker is made up of a majority of amino acids that are sterically unhindered, such as glycine and alanine.

In still further aspects, the polypeptide may be attached to a heterologous peptide or protein. Fusion proteins may include myc, HA-, or His6-tags. Fusion proteins further include the polypeptide described herein fused to the Fc domain of a human IgG. In particular aspects, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130. The Fc moiety can be derived from mouse IgG1 or human IgG2_{M}4. Human IgG2_{M}4 (See U.S. Published Application No. 20070148167 and U.S. Published Application No. 20060228349) is an antibody from IgG2 with mutations with which the antibody maintains normal pharmacokinetic profile but does not possess any known effector function.

Fusion proteins further include the polypeptide is fused to human serum albumin, transferrin, or an antibody.

In further still aspects, the polypeptide is conjugated to a carrier protein such as human serum albumin, transferrin, or an antibody molecule.

The polypeptides of the invention may be linear or cyclic (cyclization may improve stability). Cyclization may take place by any means known in the art. Where the compound is composed predominantly of amino acids, cyclization may be via N- to C-terminal, N-terminal to side chain and N-terminal to backbone, C-terminal to side chain, C-terminal to backbone, side chain to backbone and side chain to side chain, as well as backbone to backbone cyclization. Cyclization of the peptide may also take place through non-amino acid organic moieties comprised in the peptide.

The polypeptides of the present invention can be biochemically synthesized such as by using standard solid phase techniques. These methods include exclusive solid phase synthesis, partial solid phase synthesis methods, fragment condensation, classical solution synthesis. Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Large scale peptide synthesis is described by Andersson Biopolymers 2000;55(3):227-50.

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Recombinant techniques may also be used to generate the polypeptides of the present invention. To produce a polypeptide of the present invention using recombinant technology, a polynucleotide encoding the polypeptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

In addition to being synthesizable in host cells, the polypeptides of the present invention can also be synthesized using in vitro expression systems. These methods are well known in the art and the components of the system are commercially available.

As mentioned, the polypeptides of this aspect of the present invention polypeptide comprise a 20S proteasome inhibitory activity. In one embodiment, the activity which is inhibited (or down-regulated) is ubiquitin-independent proteasomal degradation. Thus, the polypeptides of the present invention may reduce the rate of degradation of proteins that are targets of the 20S proteasome. In another embodiment, the polypeptide inhibits the 20S proteasome when it is not comprised in the 26S particle. The polypeptides may inhibit the activity of the 20S proteasome of any organism (although preferably, the polypeptides are capable of inhibiting the human 20S proteasome).

The polypeptides may be capable of inhibiting the activity of the 20S proteasome when it resides inside a cell (i.e. intracellular 20S proteasome). Alternatively, or additionally, polypeptides may be capable of inhibiting the activity of the 20S proteasome when it resides in the extracellular space, such as blood plasma, the cerebrospinal and alveolar fluids as well as in culture medium conditioned by some human cell lines. The proteasomes which have been detected in normal human blood plasma are variously referred to as “circulating proteasomes” (c-proteasomes), “plasma-proteasomes” (p-proteasomes).

The 20S proteasome is a 700 kDa cylindrical-shaped multicatalytic protease complex comprised of 28 subunits organized into four rings. In yeast and other eukaryotes, 7 different alpha subunits form the outer rings and 7 different beta subunits comprise the inner rings. The alpha subunits serve as binding sites for the 19S (PA700) and 11S (PA28) regulatory complexes, as well as a physical barrier for the inner proteolytic chamber formed by the two beta subunit rings.

According to a particular embodiment, the polypeptides of this aspect of the present invention do not affect the chymotrypsin like activity of the 20S proteasome to a greater extent than the trypsin-like and/or peptidylglutamyl-peptide activities of the 20S proteasome. Methods of analyzing whether the polypeptides comprise such an inhibitory activity are known in the art and include measurements using fluorogenic peptide substrates.

Preferably, the polypeptides inhibit the activity of the 20S proteasome to a greater extent than they inhibit the activity of the 26S proteasome. For example the Ki of the polypeptide may be at least 2 fold, preferably at least 5 fold lower for the 20S proteasome than for the 26S proteasome. Methods of determining the inhibitory activity of the polypeptides towards the 26S proteasome are known in the art and include co-immunoprecipitation assays.

The polypeptides of this aspect of the present invention may bind directly to the 20S proteasome. Preferably, they do not bind the chymotrypsin-like β5 subunit of the 20S proteasome.

According to a particular embodiment, the polypeptides (for example CBR3 and NQO1) bind to the β-subunit ring of the proteasome.

According to still another embodiment, the polypeptides bind to the β5 subunit ring of the proteasome.

The instant polypeptides show selectivities for the 20S proteasome over other proteases such as cathepsins, calpains, papain, chymotrypsin, trypsin, tripeptidyl pepsidase II. The selectivities of the enzyme inhibitors for 20S proteasome are such that at concentrations below a predetermined level, the enzyme inhibitors show reduction of the degradation activity of the 20S proteasome, while not showing inhibition of the catalytic activity of other proteases such as cathepsins, calpains, papain, chymotrypsin, trypsin, tripeptidyl pepsidase II. Enzyme kinetic assays are disclosed in U.S. application Ser. No. 09/569,748, Example 2 and Stein et al., Biochem. (1996), 35, 3899-3908.

Since the polypeptides disclosed herein are capable of inhibiting the 20S proteasome, the present inventors propose that they may be used to treat and/or prevent 20S associated diseases, examples of which are provided herein below.

In general, the proteolytic activity of the 26S/20S proteasome in eukaryotes is central to a wide array of processes such as cell cycle progression, signal transduction, DNA repair, transcription, apoptosis, and angiogenesis. When aberrant, all can unleash control of cellular growth, promote tumorigenesis, and/or exacerbate malignancy. Therefore, proteasomes have become attractive targets for treating numerous cancers. Known proteasome inhibitors, bortezomib and carfilzomib bind the chymotrypsin-like β5 subunit of the 20S proteasome; thus, they inhibit both the 20S and 26S proteasomes. Selective inhibition of only the 20S proteasome is expected to provide an attractive means for expanding the range of cancers in which proteasome inhibitor therapy is effective, and reduce the deleterious side effects of current treatments.

Selective 20S proteasome inhibition is especially relevant, considering recent findings that: i) The mechanism of efficacy of proteasome inhibitors is unclear, but they are thought to stabilize I-κB, an important suppressor of NF-κB signaling. They also cause accumulation of p27 and p53, negative regulators of the cell cycle as well as pro-apoptotic proteins such as p21, NOXA and PUMA. All these proteins consist of IDRs, which make them susceptible to degradation by the 20S proteasome in a ubiquitin- independent manner. Indeed, I-κB, p53, p27 and p21 were shown to be substrates of the 20S proteasome. Thus, selective 20S inhibition will promote their cellular accumulation. ii) Conditions associated with tumorigenesis (e.g., hypoxia, matrix detachment, mitochondrial dysfunction, and inflammation) all lead to excess production of reactive oxygen species (ROS). Under such conditions, the 20S proteasome is known to be the major degradation machinery, likely due to its higher resistance to oxidation, and the sensitivity of the ubiquitinylation machinery to redox conditions. Thus, cancer cells are predicted to be more sensitive to 20S-specific inhibition than normal cells.

In addition, selective inhibition of the immune-proteasomes has also major therapeutic value, as immune-proteasomes are inappropriately expressed in human autoimmune disorders. Consequently, immune-proteasome specific inhibitors have been proposed for treatment of autoimmune disorders, as they are expected to prevent the presentation of self-antigens and reduce inflammatory cytokine secretion by immune cells.

Thus, according to another aspect of the present invention there is provided a method of treating a disease for which inhibiting a 20S proteasome is advantageous in a subject in need thereof, the method comprising administering to the subject a therapeutically effective amount of the isolated polypeptide described herein, thereby treating the disease.

According to another aspect of the present invention there is provided a method of treating a disease for which inhibiting a 20S proteasome is advantageous in a subject in need thereof, the method comprising administering to the subject a therapeutically effective amount of an isolated polypeptide comprising a CATH 3.40 architecture, said fold comprising the amino acid sequence as set forth in SEQ ID NO: 18, with the proviso that the isolated polypeptide is not full length DJ-1 or NQO1.

Examples of such polypeptides are described herein above.

According to a particular embodiment, the polypeptide is not a full length wild type protein such as DJ-1 of NQO1.

In another embodiment, the polypeptide is not a full length wild type protein such as NQO2, CBR3, PGDH, RBBP9, NRas, KRas, HRas, RhoA, RhoB, RhoC, Rap1A, Rap1B, Rap2A, ETFB or PGAM1.

Examples of cancers that may be treated using the proteasome inhibitors of this aspect of the present invention include, but are not limited to adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; triple negative breast cancer, Burkitt' s lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with eosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, malignant melanoma, meningioma; multiple endocrine neoplasia; multiple myeloma, myeloid malignancy, predisposition to; myxosarcoma, neuroblastoma; osteosarcoma; osteocarcinoma, ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, basal cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, and the like.

The formation of new blood vessels, angiogenesis, is critical for the progression of many diseases, including cancer metastases, diabetic retinopathy, and rheumatoid arthritis. Many factors associated with angiogenesis, eg, cell adhesion molecules, cytokines, and growth factors, are regulated through the proteasome, and, hence, alteration of its activity will affect the degree of vessel formation. Oikawa et al [Biochem Biophys Res Commun. 1998;246:243-248] demonstrated that a particular proteasome inhibitor, lactacystin, significantly reduced angiogenesis, suggesting that it, or related compounds, could be beneficial in disease states that rely on the formation of new blood vessels.

Thus, according to another embodiment, the disease in which inhibiting a proteasome is advantageous is an angiogenesis associated disease.

The proteasome is intimately linked to the production of the majority of the class I antigens. It is therefore conceivable that excessive inhibition of the proteasome might also increase the chance of viral infections such as HIV.

Through its regulation of NF-kappa B, the proteasome is central to the processing of many pro-inflammatory signals. Once released from its inhibitory complex through proteasome degradation of I kappa B, NF-kappa B induces the activation of numerous cytokines and cell adhesion molecules that orchestrate the inflammatory response. Thus, the present invention contemplates use of the proteasome inhibitors of the present invention for the treatment of inflammatory diseases including but not limited to asthma, ischemia and reperfusion injury, multiple sclerosis, rheumatoid arthritis, psoriasis, autoimmune thyroid disease, cachexia, Crohn disease, hepatitis B, inflammatory bowel disease, sepsis, systemic lupus erythematosus, transplantation rejection and related immunology and autoimmune encephalomyelitis.

In addition, it has been shown that blocking proteasome activity reduces neuron and astrocyte degeneration and neutrophil infiltration and therefore can be potential therapy for stroke and neurodegenerative diseases including Parkinson's disease, Multiple Sclerosis, ALS, multi-system atrophy, Alzheimer's disease, stroke, progressive supranuclear palsy, fronto-temporal dementia with parkinsonism linked to chromosome 17 and Pick's disease.

Examples of autoimmune diseases which can be treated by the polypeptides of the present invention include, but are not limited to cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998;7 Suppl 2:S135), myocardial infarction (Vaarala O. Lupus. 1998;7 Suppl 2:S132), thrombosis (Tincani A. et al., Lupus 1998;7 Suppl 2:S107-9), Wegener's granulomatosis, Takayasu's arteritis, Kawasaki syndrome (Praprotnik S. et al., Wien Klin Wochenschr 2000 Aug. 25;112 (15-16):660), anti-factor VIII autoimmune disease (Lacroix-Desmazes S. et al., Semin Thromb Hemost.2000;26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May;151 (3):178), antiphospholipid syndrome (Flamholz R. et al., J Clin Apheresis 1999;14 (4):171), antibody-induced heart failure (Wallukat G. et al., Am J Cardiol. 1999 Jun. 17;83 (12A):75H), thrombocytopenic purpura (Moccia F. Ann Ital Med Int. 1999 April-June;14 (2):114; Semple J W. et al., Blood 1996 May 15;87 (10):4245), autoimmune hemolytic anemia (Efremov D G. et al., Leuk Lymphoma 1998 January;28 (3-4):285; Sallah S. et al., Ann Hematol 1997 March;74 (3):139), cardiac autoimmunity in Chagas' disease (Cunha-Neto E. et al., J Clin Invest 1996 Oct. 15;98 (8):1709) and anti-helper T lymphocyte autoimmunity (Caporossi AP. et al., Viral Immunol 1998;11 (1):9).

Examples of autoimmune rheumatoid diseases include, but are not limited to rheumatoid arthritis (Krenn V. et al., Histol Histopathol 2000 July;15 (3):791; Tisch R, McDevitt H O. Proc Natl Acad Sci units S A 1994 Jan. 18;91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October;34 Suppl:S125), autoimmune thyroid diseases, Graves' disease (Orgiazzi J. Endocrinol Metab Clin North Am 2000 June;29 (2):339; Sakata S. et al., Mol Cell Endocrinol 1993 March;92 (1):77), spontaneous autoimmune thyroiditis (Braley-Mullen H. and Yu S, J Immunol 2000 Dec. 15;165 (12):7262), Hashimoto's thyroiditis (Toyoda N. et al., Nippon Rinsho 1999 August;57 (8):1810), idiopathic myxedema (Mitsuma T. Nippon Rinsho. 1999 August;57 (8):1759), ovarian autoimmunity (Garza K M. et al., J Reprod Immunol 1998 February;37 (2):87), autoimmune anti-sperm infertility (Diekman A B. et al., Am J Reprod Immunol. 2000 March;43 (3):134), autoimmune prostatitis (Alexander R B. et al., Urology 1997 December;50 (6):893) and Type I autoimmune polyglandular syndrome (Hara T. et al., Blood. 1991 Mar. 1;77 (5):1127).

Examples of autoimmune gastrointestinal diseases include, but are not limited to, chronic inflammatory intestinal diseases (Garcia Herola A. et al., Gastroenterol Hepatol. 2000 January;23 (1):16), celiac disease (Landau Y E. and Shoenfeld Y. Harefuah 2000 Jan. 16;138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March;54 (3):382), primary biliary cirrhosis (Jones DE. Clin Sci (Colch) 1996 November;91 (5):551; Strassburg C P. et al., Eur J Gastroenterol Hepatol. 1999 June;11 (6):595) and autoimmune hepatitis (Manns M P. J Hepatol 2000 August;33 (2):326).

Examples of autoimmune neurological diseases include, but are not limited to, multiple sclerosis (Cross A H. et al., J Neuroimmunol 2001 Jan. 1;112 (1-2):1), Alzheimer's disease (Oron L. et al., J Neural Transm Suppl. 1997;49:77), myasthenia gravis (Infante A J. And Kraig E, Int Rev Immunol 1999;18 (1-2):83; Oshima M. et al., Eur J Immunol 1990 December;20 (12):2563), neuropathies, motor neuropathies (Kornberg A J. J Clin Neurosci. 2000 May;7 (3):191); Guillain-Barre syndrome and autoimmune neuropathies (Kusunoki S. Am J Med Sci. 2000 April;319 (4):234), myasthenia, Lambert-Eaton myasthenic syndrome (Takamori M. Am J Med Sci. 2000 April;319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27;98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January;156 (1):23); dysimmune neuropathies (Nobile-Orazio E. et al., Electroencephalogr Clin Neurophysiol Suppl 1999;50:419); acquired neuromyotonia, arthrogryposis multiplex congenita (Vincent A. et al., Ann N Y Acad Sci. 1998 May 13;841:482), neuritis, optic neuritis (Soderstrom M. et al., J Neurol Neurosurg Psychiatry 1994 May;57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September;123 (1):92) and smooth muscle autoimmune disease (Zauli D. et al., Biomed Pharmacother 1999 June;53 (5-6):234).

Examples of autoimmune nephric diseases include, but are not limited to, nephritis and autoimmune interstitial nephritis (Kelly C J. J Am Soc Nephrol 1990 August;1 (2):140).

Examples of autoimmune diseases related to reproduction include, but are not limited to, repeated fetal loss (Tincani A. et al., Lupus 1998;7 Suppl 2:S107-9).

Examples of autoimmune connective tissue diseases include, but are not limited to, ear diseases, autoimmune ear diseases (Yoo T J. et al., Cell Immunol 1994 August;157 (1):249) and autoimmune diseases of the inner ear (Gloddek B. et al., Ann N Y Acad Sci 1997 Dec. 29;830:266).

Examples of autoimmune systemic diseases include, but are not limited to, systemic lupus erythematosus (Erikson J. et al., Immunol Res 1998;17 (1-2):49) and systemic sclerosis (Renaudineau Y. et al., Clin Diagn Lab Immunol. 1999 March;6 (2):156); Chan O T. et al., Immunol Rev 1999 June;169:107).

The polypeptides of the present invention may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the caspase 6 inhibitory peptides accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations, which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (caspase-6 inhibitory peptides) effective to prevent, alleviate or ameliorate symptoms of a disorder (e.g., Huntington's Disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to brain or blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

Purification of Human DJ-1 and Saccharomyces cerevisiae DJ-1 (Hsp32)

BL21(DE3) E. coli were transformed with pET-15b vector containing cDNA of human DJ-1, or pET28 vector containing cDNA of S. cerevisiae DJ-1 (Hsp32). Cells were grown in LB medium supplemented with 100 μg/ml ampicillin or 50 μg/ml kanamycin respectively, at 37° C. until they reached OD₆₀₀ 0.45. Protein expression was induced by the addition of 0.4 mM isopropyl-b-D-thiogalactoside (IPTG) for 2.5 h. Cells were harvested by centrifugation at 5000 g for 10 minutes, and resuspended in 50 ml of 50 mM Tris-HCl pH 7.4, 2 mM EDTA, 1 mM DTT, 1 mM PMSF and a protease inhibitor cocktail (Complete, Roche). Cells were lysed in a French Press, centrifuged for 10 min at 5000 g and the lysate was passed through a Source-15Q anion exchange 55 ml column (GE Healthcare) pre-equilibrated with 50 mM Tris-HCl pH 7.4, 1 mM DTT. After lysate loading, proteins were eluted with 200 ml of 50 mM Tris-HCl pH 7.4, 1 mM DTT. 50 ml fractions were collected and DJ-1-containing fractions (eluted after 150-200 ml) were concentrated using a 3-kDa Amicon Ultra column (Millipore). Concentrated DJ-1 was loaded onto a gel filtration column (Superdex 200, 10/300 GL, GE Healthcare), pre-equilibrated with 50 mM Tris-HCl pH 7.4, 300 mM NaCl and 1 mM DTT. DJ-1-containing fractions were combined, concentrated, frozen in liquid nitrogen and stored at −80° C.

Purification of Thermoplasma acidophilum DJ-1

The BL21 (DE3) strain of E. coli was transformed with a pET28a-TEVH-DJ-1 vector harboring the cDNA of T. acidophilum DJ-1 with a His-tag. Cells were grown at 37° C. to an OD600 of 0.5 in 100 ml LB medium supplemented with 50 mg/ml kanamycin. Protein expression was induced by the addition of 0.5 mM IPTG for 7 h at 37° C. and then the cells were moved to 16° C. for overnight protein expression. Cells were harvested by centrifugation for 20 min at 5000 g and resuspended in lysis buffer (50 mM Tris-HCl pH 7.5, 50 mM NaCl, 20 mM Imidazole, 250 U Benzonase (Millipore) 1 mM PMSF). Cells were lysed by sonication and the lysate was centrifuged for 30 min at 40,000 g. The supernatant was loaded on a HisTrap FF 5 ml (GE Healthcare) pre-equilibrated with binding buffer (50 mM Tris-HCl, 50 mM NaCl, 20 mM Imidazole). After lysate loading, protein was eluted with 0-100% gradient elution buffer (50 mM Tris-HCl pH 7.5, 300 mM NaCl, 500 mM Imidazole). DJ-1 containing fractions were pooled and dialyzed with TEV protease against 50 mM Tris pH 7.4, 1 mM EDTA and 2 mM DTT. Following the overnight TEV cleavage, the DJ-1 was loaded on HisTrap FF 5 ml and flow through fraction was collected, concentrated, frozen in liquid nitrogen and stored at −80° C.

### Purification of NQO2

BL21(DE3) E. coli were transformed with pET28 containing the cDNA of human NQO2. Cells were grown in LB medium supplemented with 50 μg/ml kanamycin at 37° C. until they reached OD₆₀₀ 0.6. Protein expression was induced by the addition of 1 mM IPTG for 3 h. Cells were harvested by centrifugation at 5000 g for 10 minutes, resuspended in 50 mM Tris-HCl pH 7.5, 150 mM NaCl, and 1 mM PMSF, and sonicated (40% amp, 30 sec pulses for 7.5 min). The lysed cells were centrifuged at 18000 rpm for 45 mins at 4° C. to remove cellular debris. The supernatant was applied to a HisTrapHP column (GE Healthcare) and eluted using a linear gradient to 400 mM imidazole, followed by gel filtration (Superdex 200, 10/300 GL, GE Healthcare) and ion exchange chromatography (HiTrap SP FF (GE Healthcare)). Purified His-TEV-NQO2 was further cleaved by TEV protease, and the His-TEV fragment was removed by binding to a Ni-NTA column.

### Purification of CBR3

BL21(DE3) E. coli were transformed with pNIC28Bsa4 vector containing the cDNA of human CBR3 with an N-terminal 6His tag (acquired from Addgene, #38800). Cells were grown in LB medium supplemented with 50 μg/ml kanamycin at 37° C. until they reached OD₆₀₀ 0.6. Protein expression was induced by the addition of 1 mM IPTG for 3 h. Cells were harvested by centrifugation at 5000 g for 10 minutes, and resuspended in 20 mM sodium dihydrogen phosphate pH 7.4, 20 mM Imidazole, 150 mM NaCl, 0.26 mM PMSF, 1 mM Benzamidine, 1 μg/ml Pepstatin. Cells were disrupted by the addition of 1 mg/ml lysozyme followed by rolling at 4° C. for 30 mins, and sonication (40% amp, 30 sec pulses for 7.5 min). The lysed cells were centrifuged at 18000 rpm for 45 mins at 4° C. to remove cellular debris. The supernatant was applied to a HisTrapHP column pre equilibrated in the resuspension buffer. His-CBR3 was eluted with a linear gradient to 400 mM imidazole over 40 mls. Fractions were evaluated by SDS-PAGE, and those containing His-CBR3 were pooled and incubated at room temperature for 3 hours with TEV protease (His tagged) to remove the His tag. The cleaved sample was dialysed overnight against 20 mM sodium dihydrogen phosphate pH 7.4, 150 mM NaCl, then re-applied to a HisTrapHP column to remove uncleaved protein and TEV protease. The flowthrough was collected, concentrated and applied to a Superdex 200, 10/300 GL gel filtration column pre-equilibrated in 20 mM sodium dihydrogen phosphate pH 7.4, 50 mM NaCl. Peak fractions were evaluated for purity by SDS-PAGE, those containing >95% pure CBR3 were pooled, concentrated to ˜100 μM, snap frozen in liquid N₂ and stored at −80° C.

### Purification of PGDH and RBBP9

BL21(DE3) E. coli were transformed with pET28 vector containing the cDNA of human PGDH and RBBP9 with a C-terminal 6His tag. Proteins were purified as for CBR3 with the following changes. After elution from the first HisTrapHP column, fractions containing PGDH-His or RBBP9 His were concentrated and applied to a Superdex 200, 10/300 GL gel filtration column pre-equilibrated in 20 mM sodium dihydrogen phosphate pH 7.4, 50 mM NaCl. Peak fractions were evaluated for purity by SDS-PAGE, those containing >95% pure PGDH-His or RBBP9-His were pooled, concentrated to ˜100 μM, snap frozen in liquid N₂ and stored at −80° C.

### Purification of NRas, KRas, HRas and RhoA

pET28-MHL plasmids containing N-terminally His tagged NRas (1-172), KRas (1-169) and HRas (1-172), and pNICBsa4 containing RhoA (1-184) were purchased from Addgene (#25256, #25153 (contains Q61H mutation, mutated back to WT), #55653 and #73231 respectively). BL21(DE3) E. coli were transformed with the vectors, and the proteins were expressed and purified as for CBR3 with the following changes. Cells were resuspended in 20 mM Tris-HCl pH 7.4, 20 mM Imidazole, 150 mM NaCl, 0.26 mM PMSF, 1 mM Benzamidine, 1 μg/ml Pepstatin. TEV cleaved proteins were dialysed against 20 mM Tris pH 7.4, 150 mM NaCl. The Superdex 200, 10/300 GL gel filtration column was pre-equilibrated in 20 mM Tris pH 7.4, 50 m M NaCl.

Purification of Mammalian 20S from Rat Liver

Rat livers were chosen as our source for 20S proteasomes, given the high evolutionary conservation of subunit sequences that exist between human and rat (>96% identity). Purification of the rat 20S proteasome was performed as described previously. In brief, rat livers were homogenized in buffer containing 20 mM Tris-HCl pH 7.5, 1 mM EDTA, 1 mM DTT and 250 mM sucrose. The extract was subjected to centrifugation at 1,000 g for 15 min. The supernatant was then diluted to 400 ml to a final concentration of 0.5 M NaCl and 1 mM DTT and subjected to ultracentrifugation for 2.2 h at 145,000 g. The supernatant was centrifuged again at 150,000 g for 6 h. The pellet containing the proteasomes was resuspended in 20 mM Tris-HCl pH 7.5 and loaded onto 1.8 L Sepharose 4B resin. Fractions containing the 20S proteasome were identified by their ability to hydrolyse the flurogenic peptide suc-LLVY-AMC, in the presence of 0.02% SDS. Proteasome-containing fractions were then combined and loaded onto four successive anion exchange columns: Source Q15, HiTrap DEAE FF and Mono Q 5/50 GL (GE Healthcare). Elution was performed with a 0-1-M NaCl gradient. Active fractions were combined, and buffer exchanged to 10 mM phosphate buffer pH 7.4 containing 10 mM MgCl₂ using 10 kD Vivaspin 20 ml columns (GE Healthcare). Samples were then loaded onto a CHT ceramic hydroxyapatite column (Bio-Rad Laboratories Inc.); a linear gradient of 10-400 mM phosphate buffer was used for elution. The purified 20S proteasomes were analysed by SDS-PAGE, activity assays and MS analysis.

Purification of Yeast 20S Proteasomes from S. cerevisiae

S. cerevisiae expressing FLAG-tagged 20S proteasome (Pre1) were grown in 4×700 ml YPD medium overnight at 30° C. Cells were harvested at 5000 g for 20 mins, the pellets rinsed in 10 ml water and centrifuged again at 5000 g for 20 mins. The pellet was resuspended in 100 ml lysis buffer containing 50 mM Tris-HCl pH 7.5, 150 mM NaCl, 10% glycerol, 5 mM MgCl₂, 1 mM PMSF, protease inhibitor cocktail (Complete, Roche), 250 U Benzonase (Millipore). Cells were lysed using a glass bead beater, pre-chilled with 50% glycerol and dry ice, with 1 min pulses for 7 mins total. The lysed cells were separated from the glass beads, and centrifuged at 35,000 g for 20 mins at 4° C. to remove cell debris. The supernatant was collected, and incubated with 2 ml anti-FLAG M2 affinity gel (Sigma), pre rinsed with sequential washes of lysis buffer, Glycine pH 3.5 and lysis buffer, for 1.5 hours at 4° C. while gently rotating. The beads were collected, washed sequentially with lysis buffer containing 0.2% NP40, lysis buffer, and lysis buffer containing 500 mM NaCl. The last wash was incubated on the beads for 1 h at 4° C., followed by a final wash in lysis buffer. 20S proteasomes were eluted using 500 mg/ml FLAG peptide in lysis buffer containing 15% glycerol.

Purification of Archaeal 20S Proteasomes from T. acidophilum

The α and β subunits of T. acidophilum 20S proteasome were expressed as separate fusion proteins with a TEV-cleavable His tag (α) or with a NusA-His tag (β) in E. coli BL21 (DE3) cells. Expression of both subunits was induced with the addition of 1 mM IPTG, 37° C. for 3 h (α) or for 5 h (β) at 37° C. Cells were collected by centrifugation at 5,000 g for 20 min. Cells were lysed by sonication in 50 mM sodium phosphate buffer pH 8.0, supplemented with protease inhibitors (0.5 mM benzamidine, 0.1 mg/ml pepstatin A and 0.1 uM PMSF), 0.88 mg/ml lysozyme, and 250 U Benzonase (Millipore). After centrifugation at 40,000 g for 30 min, the supernatant was loaded onto a HisTrap FF (GE Healthcare) pre-equilibrated in 50 mM sodium phosphate buffer pH 8.0, 200 mM NaCl, 10 mM imidazole. The α and β subunits were eluted in 100 mM sodium phosphate buffer pH 7.8, 300 mM imidazole. The fractions containing the fusion protein were pooled and dialyzed overnight with TEV protease against 50 mM Tris pH 7.4, 1 mM EDTA and 2 mM DTT. Following the overnight TEV cleavage, the α and β subunits were loaded onto a HisTrap FF column and flow through fractions were collected. The full proteasome (α₇β₇β₇α₇) was assembled by mixing a slight molar excess of α subunit over β subunit, and incubated at 37° C. for 6 h. The mixture was concentrated to 0.5 ml and incubated overnight at 37° C. The assembled 20S proteasome complex was loaded onto a Superdex 200 10/300 GL pre-equilibrated in 50 mM sodium phosphate buffer pH 7.5, 200 mM NaCl.

### Degradation Assays

To monitor the ability of proteins to regulate the activity of the 20S proteasome in vitro, 10 μM of the proteins or MG132 were pre-incubated with 0.1 μM of the 20S proteasome for 30 min on ice in 50 mM HEPES pH 7.5. To initiate the assay, α-synuclein was added to 1 μM, and the reaction mixtures were incubated at 37° C. 10 μ1 samples were taken every 30 min for 120 min, quenched by the addition of reducing sample buffer and snap frozen in liquid N₂. After all time points were collected, the samples were thawed, boiled for 5 min, and loaded onto a 15% SDS-PAGE gel. Gels were stained with Commassie brilliant blue, and changes in the level of α-synuclein were quantified by band densitometry using ImageJ, normalized to T₀, and plotted using GraphpadPrism.

### Native Mass Spectrometry

Nanoflow electrospray ionization MS and tandem MS experiments were conducted under non-denaturing conditions on a QToF Q-Star Elite instrument (MDS Sciex, Canada), modified for improved transmission of large non-covalent complexes, or an Q-Exactive Plus Orbitrap EMR (ThermoFisher Scientific). Before MS analysis, 20 μl of up to 100 mM sample was buffer exchanged into 0.5-1 M ammonium acetate pH 7.5, using Bio-Spin columns (Bio-Rad). Sample concentrations were determined by ultraviolet absorbance. Assays were performed in positive ion mode and conditions were optimized to enable the ionization and removal of adducts, without disrupting the non-covalent interactions of the proteins tested. In tandem MS experiments, the relevant m/z values were isolated and argon gas was admitted to the collision cell. Spectra are shown with minimal smoothing, and without background subtraction. Typically, aliquots of 2 μl of sample were electrosprayed from gold-coated borosilicate capillaries prepared in-house. The following experimental parameters were used on the QToF Q-Star Elite instrument: capillary voltage up to 1.1 kV, declustering potential up to 220 V, focusing potential up to 240 V, a second declustering potential of 15 V, collision energy of between 20 and 200 V and an MCP of 2,350 V. The following experimental conditions were used on the Q-Exactive Plus Orbitrap EMR: capillary voltage 1.7 kV, MS spectra were recorded at low resolution (5000), and the HCD cell voltage was set to 20-50 V, at trapping gas pressure setting of 3.9. For tandem MS (MS/MS) analyses, a wide isolation window of ±2000 m/z around the most intense charge state of the 20S proteasome (around 12,000 m/z) was set in the quadrupole, allowing the transmission of only high m/z species. Transmitted ions were subjected to collision induced dissociation in the HCD cell, at an accelerating voltage of 200 V, and the trapping gas pressure was set to 1.5.

### Bioinformatics

Sequences for homologues of DJ-1 and NQO1 were acquired from UniProtKB (www(dot)uniprot(dot)org) and aligned using ClustalOmega (www(dot)ebi(dot)ac(dot)uk/Tools/msa/clustalo/). Multiple sequence alignment images were generated using Espript (espript(dot)ibcp(dot)fr) and Weblogo (weblogo(dot)Berkeley(dot)edu/logo(dot)cgi). All known Rossman fold containing human proteins, as listed in the Protein Data Bank (www(dot)rcsb(dot)org) were searched for those containing the identified N-terminal amino acid motif, ((K/R)₁₋₂(V/L/I/A)₄)—SEQ ID NO: 18. Proteins larger than 100 kDa were excluded from the final list.

### Immunoprecipitation

HEK293 cells stably expressing the FLAG-β₄ subunit were plated in six 15-cm dishes (for PGDH) or three 15-cm dishes (for CBFR3), at a density of 1.5×10⁶ cells per dish and grown for 24 h. Cells were collected by trypsinization, combined, washed in PBS and resuspended in 1 ml lysis buffer for PGDH IP (10 mM HEPES pH 7.4, 10% glycerol, 10 mM NaCl, 3 mM MgCl₂, 1 mM ATP), or 1 ml lysis buffer for CBR3 IP (10 mM HEPES pH 7.4, 10 mM NaCl, 3 mM MgCl₂) and protease inhibitors (1 mM PMSF, 1 mM benzamidine, 1.4 mg/ml 1 pepstatin A). Cells were incubated on ice for 15 min and homogenized in a glass-Teflon homogenizer for 40 strokes. Lysate was cleared by centrifugation at 10,000 g for 10 min at 4° C. For IP using anti-PGDH, 1 mg protein was diluted in 700 μl lysis buffer. For IP using anti-CBR3, 1 mg was diluted in 500 μl lysis buffer. NaCl concentration was adjusted to 150 mM. Proteins were precleared using 40 μl Protein G Sepharose (GE Healthcare), for 1 h at 4° C., at a gentle rotation. The beads were discarded and the lysate was rotated overnight at 4° C. in the presence of 8 μg PGDH antibody (sc-271418, Santa Cruz) or 15 μg anti-CBR3 (sc374393, Santa Cruz). The following morning, 40 μl Protein G Sepharose beads (GE Healthcare) were added, and lysate was rotated for 2 h at 4° C. The beads were then washed three times in lysis buffer containing 150 mM NaCl and boiled in 100 μl protein sample buffer. For IP using anti-FLAG affinity gel, 1 mg protein was diluted in 500 μl lysis buffer. NaCl concentration was adjusted to 150 mM, and rotated overnight at 4° C. in the presence of 40 μl anti-FLAG M2 affinity gel (Sigma). The following morning, beads were washed three times with lysis buffer containing 150 mM NaCl and boiled in 100 μl reducing sample buffer.

### Silencing of NQO₂ and NRas

HEK293 cells were transfected with 250 pmol siNQO2 (Dharmacon, L-006334) siNRas (Dharmacon, L-003919) or non-targeting siRNA (Dharmacon, D-001206-14) using JetPrime transfection reagent (Polyplus) according to the manufacturer's instructions, for 48 h. MG132 was added to a final concentration of 10 μM for the final 3 h before harvesting with trypsin. Cell pellets were rinsed in PBS, and lysed in modified RIPA buffer (50 mM HEPES pH 7.5, 150 mM NaCl, 1% NP40, 1% Na-deoxycholate, 1 mM EDTA, 1 mM PMSF, 1 mM Benzamidine, 1.4 μg/m1 Pepstatin) for 15 min on ice. Lysed cells were centrifuged for 10 min at 10,000 g to remove cell debris. The supernatant was collected, total protein was measured by Bradford assay and the samples adjusted with reducing sample buffer. 30 μg total protein was loaded onto 15% SDS-PAGE gels, transferred to PVDF and blotted using the following antibodies: NQO2 1:200 (sc-271665, SantaCruz), NRas 1:200 (OP-25, SantaCruz), p53HRP 1:2500 (HAF1355, Biotest), Ubiquitin 1:1000 (PW0930, Enzo), Tubulin 1:10,000 (ab184613, Abcam).

### Overexpression of CBR3, NQO2, PGDH and NRas

HEK293 cells were transfected with pCDF1 vector containing cDNA of full length human CBR3, NQO2 and PGDH. 108T melanoma cells were transfected with pCDF1 vector containing cDNA of full length human NRas. Transfections were performed using JetPrime transfection reagent (Polyplus) according to the manufacturer's instructions, for 24 h. Cells were trypsinized, rinsed in PBS, and the cell pellets lysed in modified RIPA buffer (50 mM HEPES pH 7.5, 150 mM NaCl, 1% NP40, 0.25% Na-deoxycholate, 1 mM PMSF, 1 mM Benzamidine, 1.4 μg/ml Pepstatin) for 15 min on ice. Lysed cells were centrifuged for 10 min at 10,000 g to remove cell debris. The supernatant was collected, total protein was measured by Bradford assay and the samples adjusted with reducing sample buffer. 30 μg total protein was loaded onto 15% SDS-PAGE gels, transferred to PVDF and blotted using the following antibodies: CBR3 1:1000 (15619-1-AP, Proteintech), NQO2 1:200 (sc-271665, SantaCruz), PGDH 1:200 (sc-271418, Santa Cruz), NRas 1:200 (OP-25, Millipore), GFP 1:2500 (ab290, Abcam), p53HRP 1:2500 (HAF1355, Biotest), α-synuclein 1:500 (ab51252, Abcam), 20S 1:200 (sc-58417, Santa Cruz), GAPDH 1:1000 (MAB374, Millipore).

### RESULTS

DJ-1 activity is highly conserved across evolution, suggesting the essentiality of this cellular pathway.

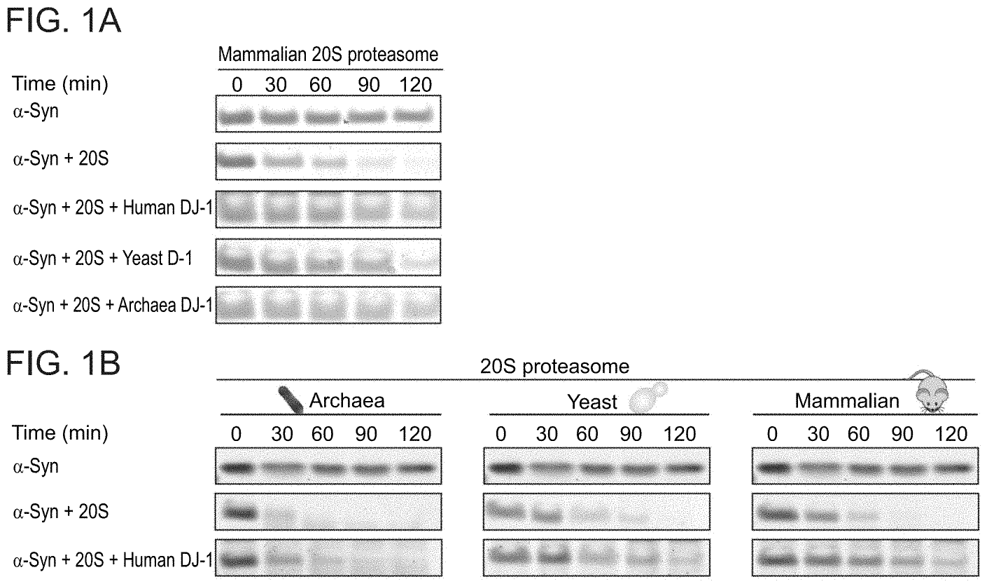

Recombinant DJ-1 orthologs from human, yeast (S. cerevisiae, Sc) and archaea (T. acidophilum, Ta) were isolated. Their ability to inhibit 20S proteasome degradation was monitored. As shown in FIG. 1A, all DJ-1 orthologs were capable of rescuing α-synuclein from proteolysis. The reciprocal experiment was then performed in which the inhibitory activity of human DJ-1 against 20S proteasomes purified from rat livers (R. norvegicus), yeast (Sc) and archaea (Ta), was examined (FIG. 1B). Results indicated that regardless of the 20S proteasome source, the presence of DJ-1 led to a vast decrease in the degradation rate.

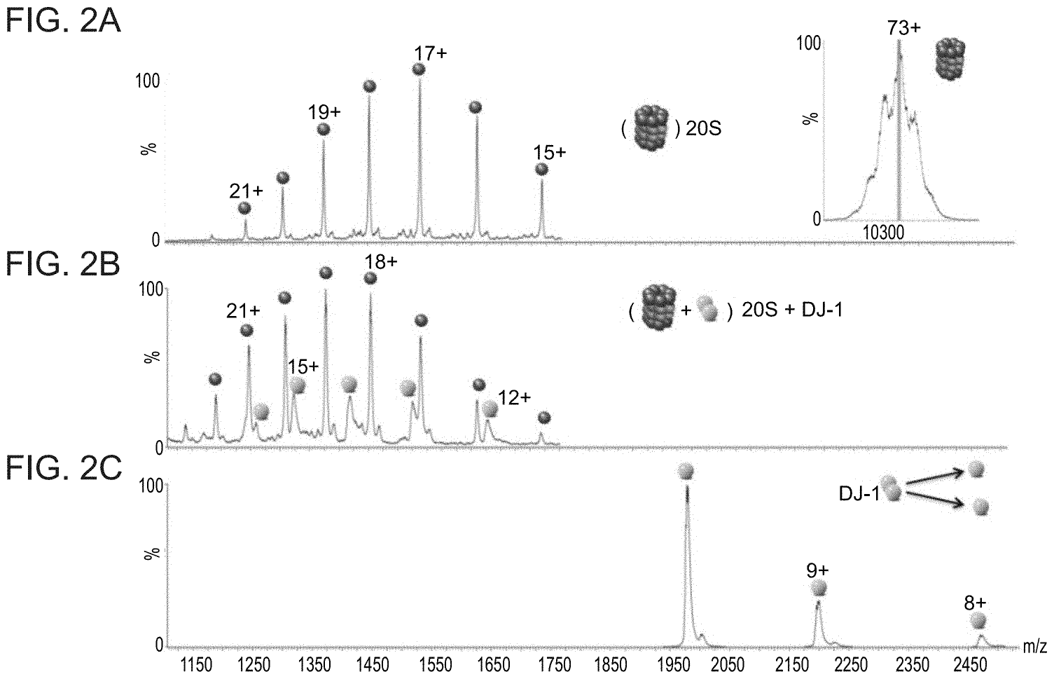

To verify these results, the present inventors examined the ability of human DJ-1 to physically bind the archaeal 20S proteasome, by mixing the 20S proteasome and DJ-1 and applying native mass spectrometry (MS) analysis; free 20S proteasome and DJ-1 were used as controls (see FIGS. 2A-C). In each of these experiments, the 20S proteasome-associated complexes appeared as a charge state series around 10,000 m/z, but the peaks were not resolved well enough to unambiguously determine whether DJ-1 was bound to 20S. Therefore, tandem MS (MS/MS) experiments were performed, in which a single peak corresponding to ions of the 20S proteasome was isolated. The ions were then subjected to collisional activation, and the individual subunits stripped from the complex, identified. The spectrum recorded for the free 20S proteasome gave rise to the dissociation of only one type of α-subunit, consistent with the architecture of this complex, in which the two homomeric α₇ ring structures are exposed (FIG. 2A). Comparison of this spectrum with that recorded for the 20S proteasome in the presence of DJ-1 revealed additional peaks that correspond in mass to the monomeric form of DJ-1 (FIG. 2B). A control MS/MS spectrum of free DJ-1 also displayed peaks corresponding in mass to monomeric DJ-1 (FIG. 2C). By extrapolation, it can be concluded that, prior to the MS/MS analysis, human DJ-1 bound the archaeal 20S proteasome. Taken together, the present results indicate that the DJ-1 activity is highly conserved across evolution, highlighting the essential nature of this cellular process.

### Identifying a Highly Conserved N-Terminal Segment Based on the High Functional Conservation of DJ-1

One of the first characterized regulators of the 20S proteasome is a cytosolic antioxidant enzyme known as NAD(P)H:quinone-oxidoreductase-1 (NQO1). This protein directly binds the 20S proteasome, and rescues key regulatory proteins such as p53, p73α and c-Fos from 20S proteolysis. Both NQO1 and DJ-1 are homodimers and comprise a CATH 3.40 architecture. Both are involved in the cellular defense mechanism against oxidative stress, and their expression levels are increased in several types of cancer. Moreover, NQO1 and DJ-1 are linked to neurodegenerative diseases: in particular, mutations in NQO1 lead to an increased risk of Alzheimer's disease, while mutations in DJ-1 are linked to familial Parkinson's disease.

The present inventors performed a bioinformatic search to identify putative sequence motifs that are common to both DJ-1 and NQO1. More specifically, sequences of DJ-1 and NQO1 homologues from 36 different species, including those from archaea, bacteria, yeast, plants, fish and mammals, were aligned using ClustalOmega. Multiple sequence alignments revealed a highly conserved motif (MX_{1,4}(K/R)₁₋₂(V/L/I/A)₄)—SEQ ID NO: 19, located at the N-terminal of the two proteins, consisting of positively charged residues followed by a short stretch of hydrophobic residues.

Discovering a Novel Family of 20S Proteasome Inhibitory Proteins (20s PIPs, also Referred to Herein as Catalytic Core Regulators (CCRs)

The identified sequence motif was then used to search the human proteome for other proteins that share this motif, of which particular emphasis was placed on those proteins already classified as CATH 3.40 architecture containing proteins. In total, 17 candidates have now been identified as potential new 20S regulatory proteins (Table 3). Interestingly, many of these proteins have already been characterized as enzymes in a variety of other pathways and play roles in cancer progression and development. In addition to DJ-1 and NQO1, 6 proteins from the list were expressed and purified and subjected to analysis by the in vitro degradation assays to assess their ability to inhibit the 20S proteasome mediated degradation of α-synuclein (FIGS. 3A-B). Strikingly, they were all shown to inhibit the 20S proteasome.

TABLE 3

| Gene | Protein | N-terminal | Size | |
|---|---|---|---|---|
| name | name | sequence | (kDa) | PDB |
| PARK7 | DJ-1 | MASKRALVIL | 20 | 1P5F |
| | (Uniprot | SEQ ID NO: 1 | | |
| | No. Q99497) | | | |
| NQO1 | NQO1 | MVGRRALIV | 31 | 1D4A |
| | (Uniprot | SEQ ID NO: 2 | | |
| | No. P15559) | | | |
| NQO2 | NQ02 | MAGKKVLIV | 26 | 1QR2 |
| | (Uniprot | SEQ ID NO: 3 | | |
| | No. P16083) | | | |
| CBR3 | CBR3 | MSSCSRVALV | 31 | 2HRB |
| | (Uniprot | SEQ ID NO: 4 | | |
| | No. O75828) | | | |
| PGDH | PGDH | MHVNGKVALV | 30 | 2GDZ |
| | (Uniprot | SEQ ID NO: 5 | | |
| | No. P15428) | | | |
| RBBP9 | RBBP9 | MASPSKAVIV | 21 | 2QS9 |
| | (Uniprot | SEQ ID NO: 6 | | |
| | No. O75884) | | | |
| RASN | NRas | MTEYKLVVV | 21 | 3CON |
| | (Uniprot | SEQ ID NO: 7 | | |
| | No. P01111) | | | |
| RASK | KRas | MTEYKLVVV | 22 | 4IPK |
| | (Uniprot | SEQ ID NO: 8 | | |
| | No. P01116) | | | |
| RASH | HRas | MTEYKLVVV | 21 | 4Q21 |
| | (Uniprot | SEQ ID NO: 9 | | |
| | No. P01112) | | | |
| RHOA | RhoA | MAAIRKKLVIV | 22 | 1FTN |
| | (Uniprot | SEQ ID NO: 10 | | |
| | No. P61586) | | | |
| RHOB | RhoB | MAAIRKKLVVV | 22 | 2FV8 |
| | (Uniprot | SEQ ID NO: 11 | | |
| | No. P62745) | | | |
| RHOC | RhoC | MAAIRKKLVIV | 22 | 2GCN |
| | (Uniprot | SEQ ID NO: 12 | | |
| | No. P08134) | | | |
| RAP1A | Rap1A | MREYKLVVL | 21 | 4KVG |
| | (Uniprot | SEQ ID NO: 13 | | |
| | No. P62834) | | | |
| RAP1B | Rap1B | MREYKLVVL | 21 | 3X1W |
| | (Uniprot | SEQ ID NO: 14 | | |
| | No. P61224) | | | |
| RAP2A | Rap2A | MREYKVVVL | 21 | 1KA0 |
| | (Uniprot | SEQ ID NO: 15 | | |
| | No. P10114) | | | |
| ETFB | ETFB | MAELRVLVAV | 28 | 1EFV |
| | (Uniprot | SEQ ID NO: 16 | | |
| | No. P38117) | | | |
| PGAM1 | PGAM1 | MAAYKLVLI | 29 | 4GPI |
| | (Uniprot | SEQ ID NO: 17 | | |
| | No. P18669) | | | |

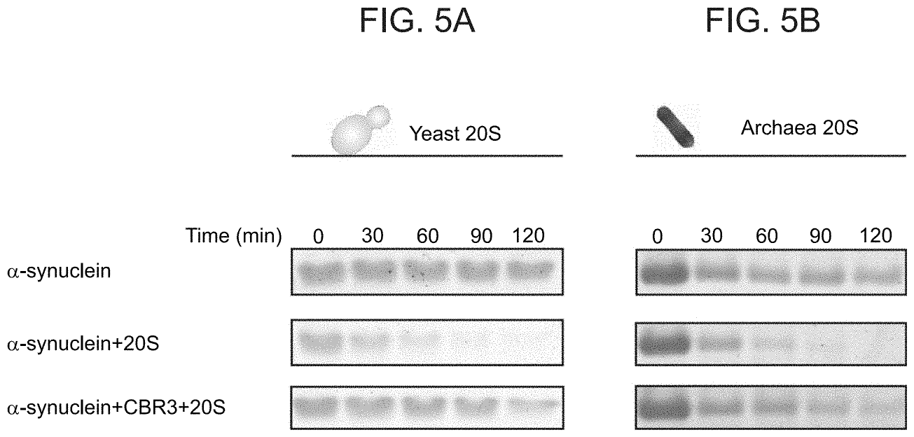

To determine whether the 20S proteasome inhibitory proteins (PIPs) identified herein inhibit proteolysis by physical interactions with the 20S proteasome, native MS was employed. Binding was determined by isolating the peak series corresponding to the intact 20S proteasome in the mass spectrometer, increasing the collision energy and monitoring the dissociation of proteasome subunits and associated proteins. Representative data for CBR3 and NRas, confirming their ability to physically bind the 20S proteasome, is shown in FIGS. 4A-C. In addition, the ability of the human proteins to inhibit the archaeal 20S proteasome was preserved, as demonstrated for CBR3 (FIGS. 5A-B), highlighting the conservation and essentiality of this process.

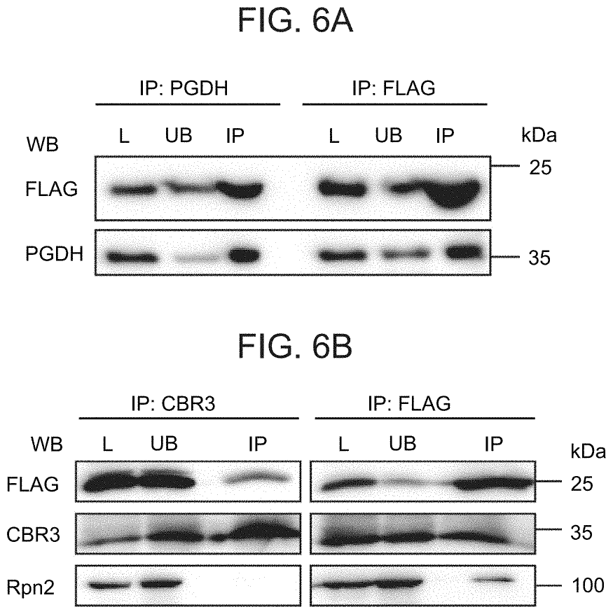

To further validate the results, immunoprecipitation experiments were performed, using a FLAG-tagged β4 subunit of the 20S proteasome, expressed in HEK 293T cells. Cellular extracts were immunoprecipitated (IP) with anti-FLAG and anti-PGDH antibodies: the precipitated material was resolved by electrophoresis, and probed with anti-PGDH, anti-CBR3 and anti-FLAG antibodies. As shown in FIGS. 6A-B, the reciprocal co-IP experiments confirmed the interaction between the 20S proteasome and endogenous PGDH and CBR3. Taken together, the present results demonstrate that by identifying the basic sequence and structural elements that are required for 20S proteasome inhibition, a new class of 20S PIPs has been discovered.

### Cellular Levels of 20S Proteasome Substrates

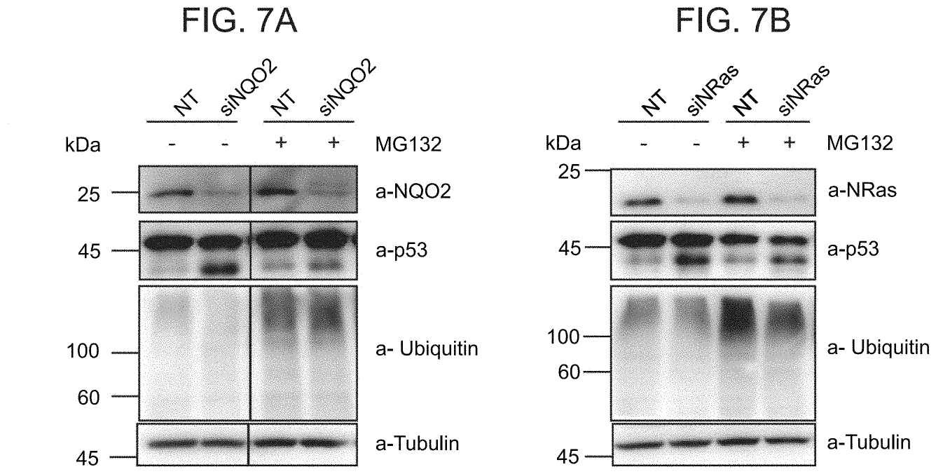

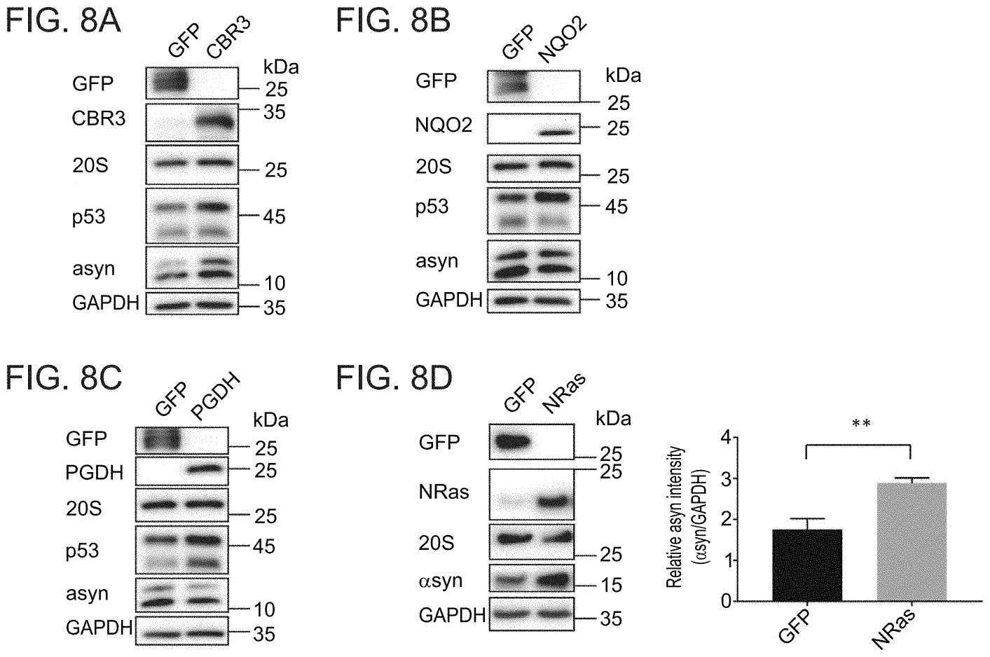

To validate the in vitro observations, NQO2 and NRas were silenced in HeK293 cells and the effect on the levels of p53, a 20S proteasome substrate was tested (FIGS. 7A-B). Western blot analysis indicated that the decrease in NQO2 and NRas levels is correlated with an increase in Δ40p53 levels. Considering that Δ40p53 is a dominant negative p53 isoform, which is generated by 20S proteasome cleavage, the results indicate that the activity of the 20S proteasome is enhanced upon reduction of NQO2 and NRas cellular levels. In addition, overexpression of CBR3, NQO2 and PGDH in HEK293 cells, and overexpression of NRas in 108T melanoma cells, was performed (FIGS. 8A-D). Western blot analysis demonstrated that in the presence of CBR3, NQO2 and PGDH, the cellular levels of full-length p53 was stabilized, indicating inhibition of the 20S proteasome. In addition, in the presence of overexpressed CBR3 (FIG. 8A) and NRas (FIG. 8D), the levels of α-synuclein were also increased, further demonstrating inhibition of the 20S proteasome in a cellular context. These findings support the view that the identified 20S PIPs inhibit 20S proteasome activity.

### Identification of the Site of Binding to the 20S Proteasome

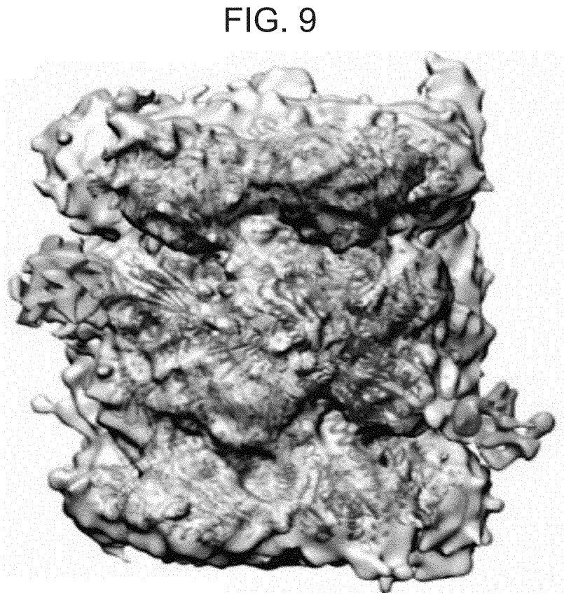

Cryo-electron microscopy (Cryo-EM) results at 7.5 Å, indicate that the Catalytic Core Regulator (CCR) CBR3, inhibits the 20S proteasome by binding to the β-subunit ring. FIG. 9 illustrates that CBR3 binds to a β-subunit of the proteasome and attenuates the catalytic sites, thus reducing the proteolytic capacity of the complex.

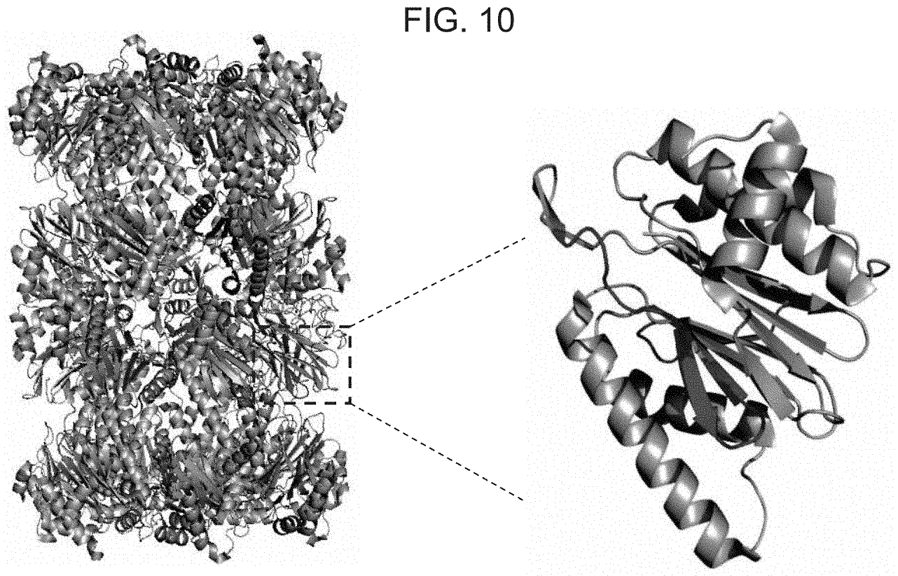

A peptide array screen indicates that the CCRs bind the β-subunit ring of the 20S proteasome. In this assay a peptide chip consisting of overlapping T. acidophilum (archaeal) 20S proteasome peptides was reacted with CBR3 and NQO1. Both CCRs bound to a sequence stretch that exists only in the β-subunit (see FIG. 10).

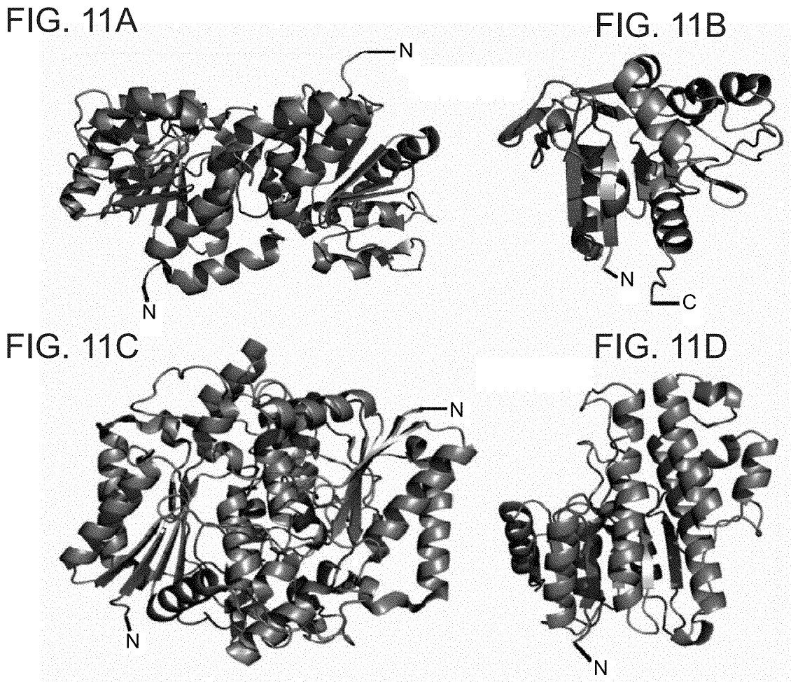

In an additional peptide array screening experiment, the present inventors reacted a peptide array chip comprising peptides of DJ-1 from both archaea and humans, and human NOQ1 and CBR3 proteins, with 20S proteasomes isolated from archaea, yeast and human cells. The resultant data indicates that all 20S proteasome species consensually bind a β-strand buried within the β-sheet core of the Rossmann fold, suggesting that the regulators undergo rearrangements upon binding to the 20S proteasome (FIGS. 11A-D).
CCRs are stable in the presence of the 20S proteasome and they do not act as competitor substrates.

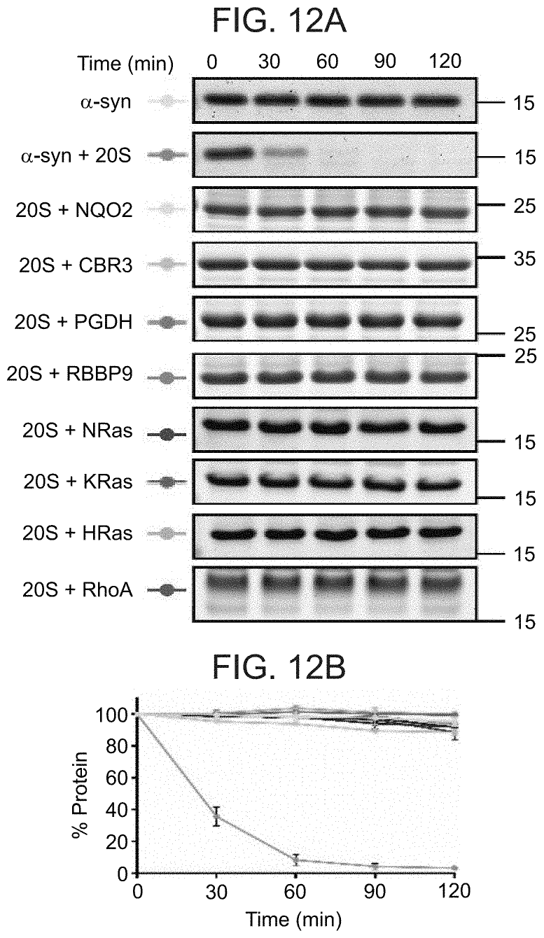

To clarify whether the inhibition is a result of competitive inhibition i.e. the CCRs themselves are being degraded by the 20S proteasome in preference to the model substrates, each CCR was analyzed by in vitro degradation assay with 20S proteasome in the absence of substrate. Quantification of the amount of CCR remaining over the course of the assay indicated that they themselves are not being degraded by the 20S proteasome, and are therefore not acting as competitive inhibitors (FIGS. 12A-B).

CCRs do not protect 20S substrates from degradation by binding to them.

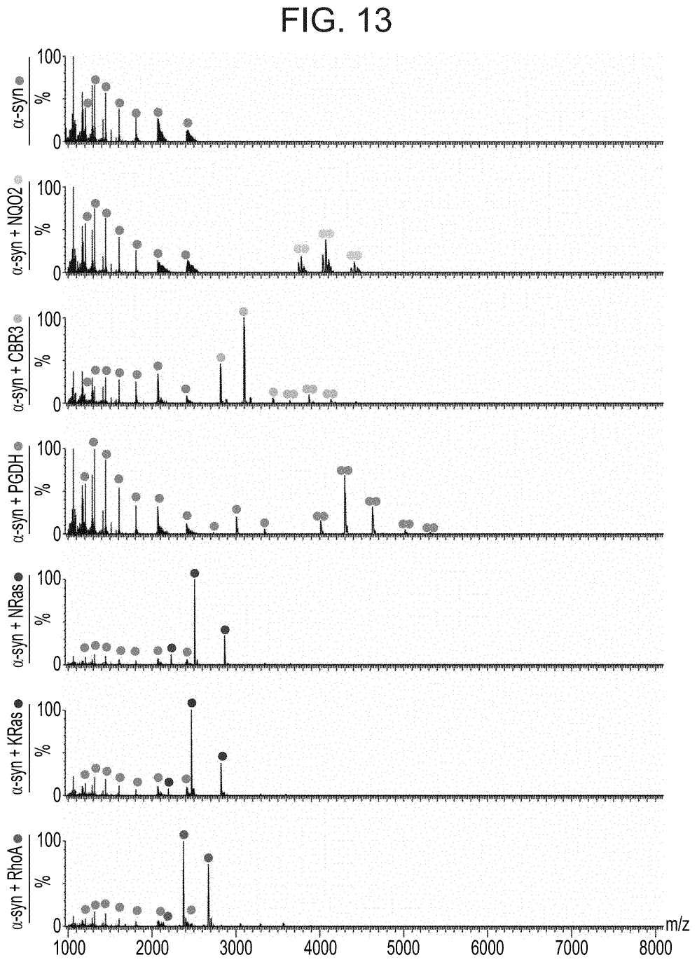

The ability of the CCRs to inhibit protein degradation could be due to either direct interactions with the 20S proteasome, or sequestration of the substrate away from the proteasome by forming a stable complex with the regulator. The present inventors therefore applied a native mass spectrometry (MS) approach to determine whether the CCRs could bind to the substrates themselves. To this end, α-synuclein was incubated with each of the CCRs and their spectra analyzed. No larger complexes were detected for any of the combinations, indicating that the inhibition of protein degradation does not occur by substrate sequestration (FIG. 13).

Systematic analysis indicated that CCRs bind directly to the 20S proteasome.

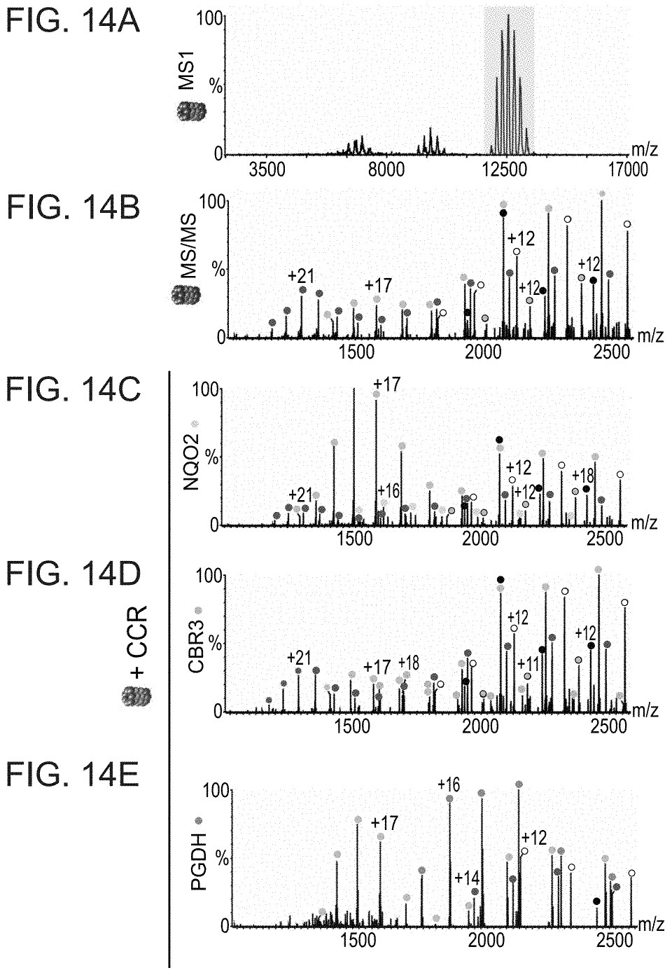

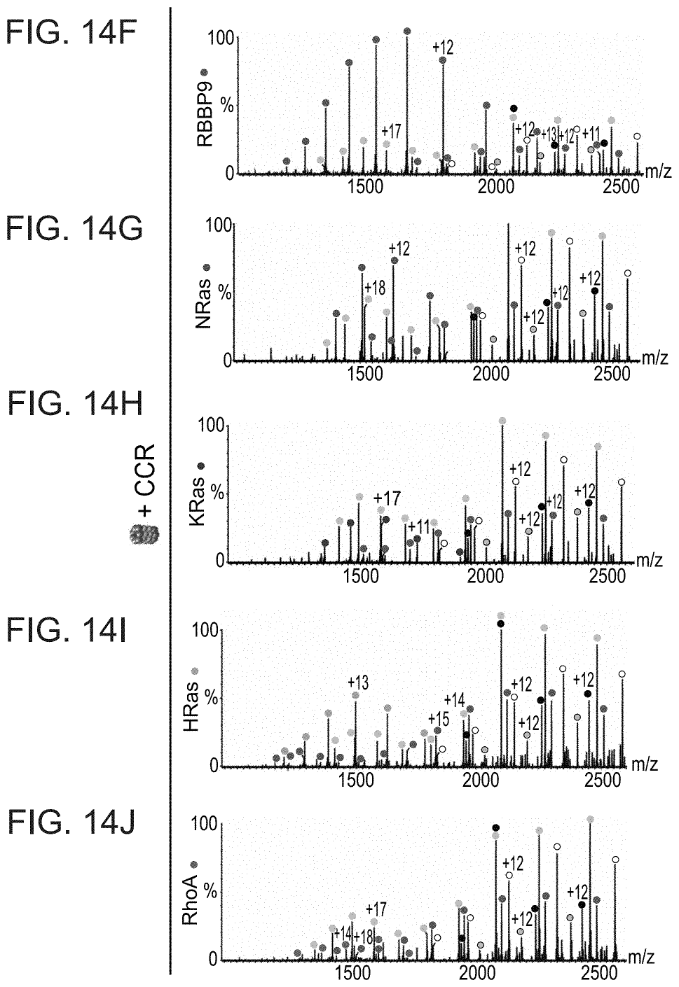

The inhibition of protein degradation is likely mediated by direct binding of CCRs to the 20S proteasome. To test this, each of the CCRs were incubated with 20S proteasome, and tandem MS (MS/MS) was employed to detect binding. MS/MS involves three stages, beginning with the acquisition of a native MS spectrum of the intact protein complexes in the protein mixture. This allows for the identification of the 20S proteasome in the high m/z range, as well as free CCR in the low m/z range. The peak series corresponding to the 20S proteasome complex is then isolated, allowing for specific selection of the 20S proteasome and its associated proteins, and not free CCR that remains unbound. The isolated complexes are subjected to high collision energies, leading to dissociation of any bound proteins as well as individual subunits of the 20S proteasome. These dissociated monomeric subunits and proteins can be detected in the low m/z range of the spectrum, and mass assignment allows for the identification of known 20S subunits, as well as CCRs that were bound to the 20S proteasome. For each of the samples containing the CCRs, a unique series of peaks corresponding in size to the predicted molecular weight of each protein were identified, that were not found in the spectrum for the 20S proteasome alone, alongside peak series corresponding to known 20S proteasome subunits (FIGS. 14A-J). This indicates that the CCRs bind directly to the 20S proteasome to regulate its function.

Immunoprecipitation assays validate that CCR directly bind the 20S proteasome.

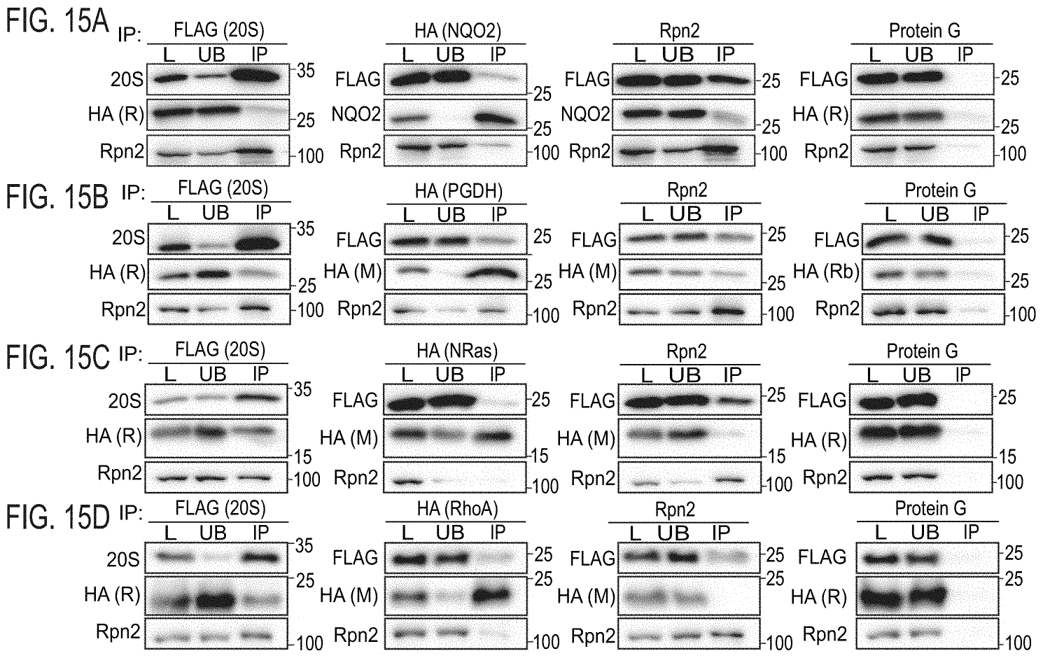

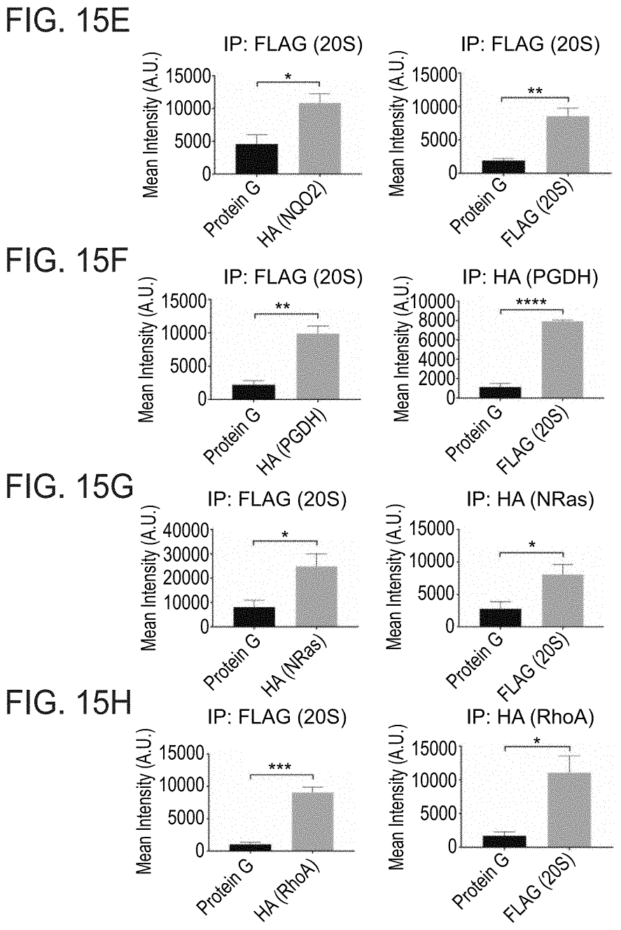

To determine whether CCR binding is specific for the 20S proteasome, or if the CCRs can also bind to the 26S proteasome, immunoprecipitation experiments were performed using HEK293 cells stably expressing the 20S proteasome β₄ subunit with a FLAG-tag on the C-terminus, in which HA-tagged CCRs were transiently overexpressed. Whole cell lysates were immunoprecipitated with either anti-FLAG, anti-Rpn2 or anti-HA antibodies, to pull down the 20S proteasome, the 26S proteasome or CCRs respectively. Bound proteins were eluted, resolved by SDS-PAGE and detected by Western blotting with anti-al (20S proteasome), anti-Rpn2 and anti-CCR antibodies. The 20S proteasome was able to pull down the 4 CCRs tested, as illustrated in FIGS. 15A-D. The reciprocal experiment demonstrated that the CCRs themselves are able to pull down the 20S proteasome. The RPN2 antibody efficiently pulled down the 20S proteasome, but a weak band was observed for several of the CCRs. These findings may suggest that CCRs can bind to 26S proteasomes that are singly capped with the 19S particle (20S-19S), consisting of an exposed 20S proteasome interface. Altogether, these results establish that the CCRs specifically bind to the 20S proteasome in cells.

CCRs exhibit differential ability to protect different substrates from degradation.

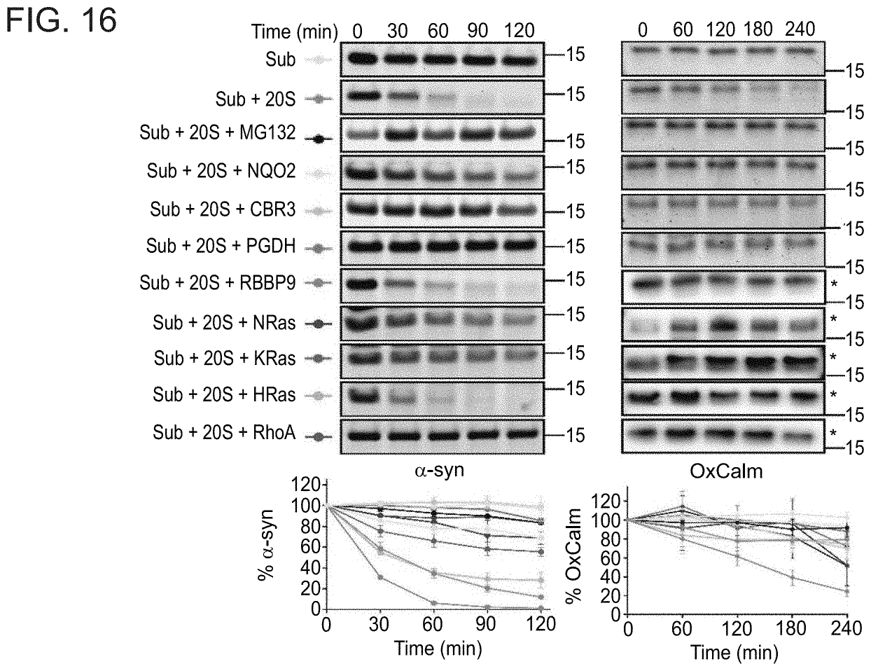

To analyze the ability of CCRs to protect substrates from 20S proteasome mediated degradation, in vitro degradation assays were performed with purified mammalian 20S proteasomes and two different model substrates, a-synuclein and oxidized calmodulin (OxCalm). MG132 was included as a control for proteasome inhibition. As illustrated in FIG. 16, the majority of the CCRs successfully inhibited the degradation of α-synuclein, with the exception of RBBP9 and HRas, while OxCam degradation was inhibited by all the candidates. These results indicate that these CCRs are capable of inhibiting protein degradation by the 20S proteasome in vitro, with an element of substrate specificity apparent between the different regulators.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An isolated recombinant polypeptide being a C-terminal truncation mutant of a protein selected from the group consisting of DJ-1, NQO1, NQO2, CBR3, PGDH, RBBP9, NRas, KRas, HRas, RhoA, RhoB, RhoC, RaplA, Rap1B, Rap2A, ETFB and PGAM1, the polypeptide capable of specifically inhibiting the activity of a 20S proteasome.

2. An isolated recombinant polypeptide comprising a CATH 3.40 architecture, said architecture comprising an amino acid sequence as set forth in SEQ ID NO: 18, wherein the polypeptide is no longer than 250 amino acids, the polypeptide capable of specifically inhibiting the activity of a 20S proteasome.

3. The isolated polypeptide of claim 2, being a C-terminal truncation mutant of a protein selected from the group consisting of DJ-1, NQO1, NQO2, CBR3, PGDH, RBBP9, NRas, KRas, HRas, RhoA, RhoB, RhoC, RaplA, Rap1B, Rap2A, ETFB and PGAM1.

4. The isolated polypeptide of claim 3, wherein the polypeptide is truncated at the C-terminus by at least 100 amino acids.

5. The isolated polypeptide of claim 1, being no longer than 300 amino acids.

6. The isolated polypeptide of claim 1, comprising a modification such that is shows enhanced bioavailability and/or efficacy in vivo as compared to the same polypeptide lacking said modification.

7. The isolated polypeptide of claim 1, being attached to a heterologous polypeptide.

8. The isolated polypeptide of claim 7, wherein said heterologous polypeptide is selected from the group consisting of human serum albumin, immunoglobulin and transferrin.

9. The isolated polypeptide of claim 2, wherein said architecture comprises a sequence selected from the group consisting of 1-17.

10. The isolated polypeptide of claim 1, being a C-terminal truncation mutant of a protein selected from the group consisting of NQO2, CBR3, PGDH, RBBP9, NRas, KRas, HRas, RhoA, RhoB, RhoC, Rap1A, Rap1B, Rap2A, ETFB and PGAM1.

11. The isolated polypeptide of claim 1, being capable of binding to said 20S proteasome.

12. An isolated polynucleotide encoding the polypeptide of claim 1.

13. A method of treating a disease for which inhibiting a 20S proteasome is advantageous in a subject in need thereof, the method comprising administering to the subject a therapeutically effective amount of the isolated polypeptide of claim 1, thereby treating the disease.

14. A method of treating a disease for which inhibiting a 20S proteasome is advantageous in a subject in need thereof, the method comprising administering to the subject a therapeutically effective amount of an isolated polypeptide comprising a CATH 3.40 architecture, said architecture comprising the amino acid sequence as set forth in SEQ ID NO: 18, with the proviso that the isolated polypeptide is not full length DJ-1 or NQO1.

15. The method of claim 14, wherein said disease is selected from the group consisting of cancer, an autoimmune disease and a neurodegenerative disease.

16. The method of claim 13, wherein said disease is selected from the group consisting of cancer, an autoimmune disease and a neurodegenerative disease.


SEQUENCE LISTING (19 sequences)

SEQ ID NO: 1 - protein, 10 aa, Artificial sequence
  polypeptide  Sequence  capable of inhibiting the 20S proteasome
MASKRALVIL

SEQ ID NO: 2 - protein, 9 aa, Artificial sequence
  polypeptide  Sequence  capable of inhibiting the 20S proteasome
MVGRRALIV

SEQ ID NO: 3 - protein, 9 aa, Artificial sequence
  polypeptide  Sequence  capable of inhibiting the 20S proteasome
MAGKKVLIV

SEQ ID NO: 4 - protein, 10 aa, Artificial sequence
  polypeptide  Sequence  capable of inhibiting the 20S proteasome
MSSCSRVALV

SEQ ID NO: 5 - protein, 10 aa, Artificial sequence
  polypeptide  Sequence  capable of inhibiting the 20S proteasome
MHVNGKVALV

SEQ ID NO: 6 - protein, 10 aa, Artificial sequence
  polypeptide  Sequence  capable of inhibiting the 20S proteasome
MASPSKAVIV

SEQ ID NO: 7 - protein, 9 aa, Artificial sequence
  polypeptide  Sequence  capable of inhibiting the 20S proteasome
MTEYKLVVV

SEQ ID NO: 8 - protein, 9 aa, Artificial sequence
  polypeptide  Sequence  capable of inhibiting the 20S proteasome
MTEYKLVVV

SEQ ID NO: 9 - protein, 9 aa, Artificial sequence
  polypeptide  Sequence  capable of inhibiting the 20S proteasome
MTEYKLVVV

SEQ ID NO: 10 - protein, 11 aa, Artificial sequence
  polypeptide  Sequence  capable of inhibiting the 20S proteasome
MAAIRKKLVIV

SEQ ID NO: 11 - protein, 11 aa, Artificial sequence
  polypeptide  Sequence  capable of inhibiting the 20S proteasome
MAAIRKKLVVV

SEQ ID NO: 12 - protein, 11 aa, Artificial sequence
  polypeptide  Sequence  capable of inhibiting the 20S proteasome
MAAIRKKLVIV

SEQ ID NO: 13 - protein, 9 aa, Artificial sequence
  polypeptide  Sequence  capable of inhibiting the 20S proteasome
MREYKLVVL

SEQ ID NO: 14 - protein, 9 aa, Artificial sequence
  polypeptide  Sequence  capable of inhibiting the 20S proteasome
MREYKLVVL

SEQ ID NO: 15 - protein, 9 aa, Artificial sequence
  polypeptide  Sequence  capable of inhibiting the 20S proteasome
MREYKVVVL

SEQ ID NO: 16 - protein, 10 aa, Artificial sequence
  polypeptide  Sequence  capable of inhibiting the 20S proteasome
MAELRVLVAV

SEQ ID NO: 17 - protein, 9 aa, Artificial sequence
  polypeptide  Sequence  capable of inhibiting the 20S proteasome
MAAYKLVLI

SEQ ID NO: 18 - protein, 6 aa, Artificial sequence
  polypeptide  Sequence  capable of inhibiting the 20S proteasome
  MISC_FEATURE (1)..(1): X can be Arg or Lys
  MISC_FEATURE (2)..(2): X can be Arg or Lys or absent
  MISC_FEATURE (3)..(6): X can be Val, Leu, Ile or Ala
XXXXXX

SEQ ID NO: 19 - protein, 11 aa, Artificial sequence
  polypeptide  Sequence  capable of inhibiting the 20S proteasome
  MISC_FEATURE (2)..(2): Xaa can be any naturally occurring amino acid
  MISC_FEATURE (3)..(5): Xaa can be any naturally occurring amino acid or absent
  MISC_FEATURE (6)..(6): X can be Arg or Lys
  MISC_FEATURE (7)..(7): X can be Arg or Lys or absent
  MISC_FEATURE (8)..(11): X can be Val, Leu, Ile or Ala
MXXXXXXXXXX